(19) United States
(12) Patent Application Publication
(10) Pub. No.: US 2010/0135963 A1
(43) Pub. Date: Jun. 3, 2010

(54) USE OF PAX4 IN PANCREATIC CELL PROLIFERATION

(76) Inventors: Benoit Raymond Gauthier, Geneve (CH); Thierry Brun, Vessy (CH); Claes Benedict Wollheim, Vessy (CH); Roland Wehr, Gottingen (DE)

Correspondence Address: FULBRIGHT & JAWORSKI L.L.P., 600 CONGRESS AVE., SUITE 2400, AUSTIN, TX, 78701, US

(21) Appl. No.: 11/573,518

(22) PCT Filed: Aug. 9, 2005
(86) PCT No.: PCT/EP05/08653

§ 371 Date: Feb. 9, 2007

Related U.S. Application Data
(60) Provisional application No. 60/600,704, filed on Aug. 11, 2004.

(30) Foreign Application Priority Data
Dec. 15, 2004 (EP) 04029743.4

(51) Int. Cl. A61K 35/12; C12N 5/00; A61P 3/10; C12N 15/63

(52) U.S. Cl. 424/93.7; 435/375; 435/325; 435/320.1

(57) ABSTRACT

The present invention relates to an in vitro method for the generation and isolation of pancreatic β-cells, comprising the steps of (a) contacting an adult cell derived from mammalian pancreatic islets or an explant culture of adult pancreatic islets with functional, wild-type Pax4; and (b) detecting and isolating; from said adult cell or explant culture, β-cells that proliferate in response to the contact with Pax4. Furthermore, the use of functional; wild-type Pax4 for the preparation of a pharmaceutical composition for transplantation and/or tissue replacement is described.

### BACKGROUND OF THE INVENTION

I. Field of the Invention

The present invention relates to an in vitro method for the generation and isolation of pancreatic β-cells, comprising the steps of (a) contacting an adult cell derived from mammalian pancreatic islets or an explant culture of adult pancreatic islets with functional, wild-type Pax4; and (b) detecting and isolating, from said adult cell or explant culture, β-cells that proliferate in response to the contact with Pax4. Furthermore, the use of functional, wild-type Pax4 for the preparation of a pharmaceutical composition for transplantation and/or tissue replacement is described.

II. Related Art

Plasma glucose levels are regulated by the action of insulin, a hormone that is produced and secreted by the pancreatic β-cells in response to nutrients. Diabetes mellitus, which comprises a heterogeneous group of hyperglycemic disorders, results from inadequate mass and function of pancreatic β-cells. Worldwide prevalence figures give an estimate of 151 million cases in 2000 and an extrapolated number of 221 million in 2010 (Zimmet, 2001). There are two forms of diabetes: type 1 diabetes is linked to selective autoimmune destruction of pancreatic β-cells while type 2 diabetes is a severe disease of intermediary metabolism usually caused by both β-cell dysfunction and resistance to the biological actions of insulin on its main target tissues (liver, muscle and fat) (Bell and Polonsky, 2001; Saltiel and Kahn, 2001). Uncontrolled elevated plasma levels of glucose as well as dyslipidemia increase the risk for diabetic complications such as cardiovascular and cerebrovascular disease, kidney disease, neuropathy and blindness. Current therapy for type 2 diabetes includes modification of life style, such as diet and exercise and the use of pharmacological agents that stimulate insulin secretion, decrease hepatic glucose production and increase sensitivity of target tissues to insulin. Nonetheless often type 2 diabetics, like type 1 patients, require treatment with insulin.

The susceptibility for type 2 diabetes is inherited but single diabetes-linked genes have only been identified in about 5% of cases. These monogenic subforms are maturity onset diabetes of the young (MODY) and mitochondrial diabetes (MD). The latter is due to mutations in the mitochondrial genome (Maassen, 2001; Maechler and Wollheim, 2001) and is associated with a selective impairment of glucose-stimulated insulin secretion (Brandle, 2001). The MODY phenotype is characterized by autosomal dominant transmission with early onset (<25 years) and primary β-cell dysfunction. Mutations in glucokinase, the rate-limiting enzyme for glucose metabolism in the β-cell, lead to MODY2 (Froguel, 1993). Interestingly, all of the additional 5 MODY subforms have been linked to mutations in genes encoding transcription factors: MODY1, hnf-4α, MODY3, hnf-1α, MODY4, ipf1/pdx1; MODY5, MODY6, Beta2/NeuroD (Kristinsson, 2001; Malecki, 1999; Ryffel, 2001; Stoffers, 1998; Mitchell, 2002). A limited number of studies have also implicated HNF-1α and Ipf1/Pdx1 in the polygenic, late onset type 2 diabetes (Hansen, 2000; Weng, 2001). Consistent with the importance of transcription factors in the development of type 2 diabetes, several recent studies have identified the paired box homeodomain family member, Pax4, as an additional predisposing gene for type 2 diabetes in the Japanese population. Homozygosity for the Pax4 mutation, located in the paired DNA binding domain, resulted in severe diabetes while heterozygous subjects were glucose intolerant (Kanatsuka, 2002; Shimajiri, 2001; Shimajiri, 2003). In contrast, no evidence of linkage between mutations in the pax4 gene and type 2 diabetes was apparent in either Ashkenazi Jews or in the French population (Dupont, 1999; Tao, 1998). The discrepancies among these studies also encountered for NeuroD may be derived from ethnic or racial differences and will require further clarification. Unfortunately, the reports segregating NeuroD with diabetes give no information on Pax4 function, which as been suggested to be controlled by NeuroD (Smith, 2000). In addition to its potential implication in type 2 diabetes, several Pax4 haplotypes have recently been associated with type 1 diabetes in Scandinavian families (Holm et al., 2004).

Increased insulin requirements in pregnancy, obesity and other insulin resistant states are compensated by β-cell hyperplasia and hypertrophy. This β-cell plasticity reaches remarkable levels in animal models such as fa/fa rats, ob/ob mice and in the liver insulin receptor null mice (Chan, 1999; Hellman, 1965; Michael, 2000). An increase in β-cell mass is also observed in human obesity (Butler, 2003; Kloppel, 1985). Up to 20% of such individuals develop type 2 diabetes probably caused by defective β-cell adaptation due to increased sensitivity to harmful environmental factors such as free fatty acids combined with predisposing genetic factors (Kashyap, 2003). There is however only limited information regarding factors controlling β-cell plasticity.

The mechanism, by which Pax4 affects β-cell function, and thus its potential implication in type 2 diabetes, is still poorly understood. In contrast to the widespread embryonic expression of other pax family members, expression of Pax4 is tightly regulated during development. Pax4 mRNA is detected in the pancreatic bud as early as mouse embryonic day 10.5 (E10.5), but expression becomes progressively restricted to the β- and δ-cells of the islet of Langerhans, producing respectively insulin and somatostatin. Although initial investigations suggested that Pax4 was not expressed in mature endocrine cells, more recent studies have detected mRNA for the transcription factor in adult human, rat and mouse pancreatic islets (Dohrmann, 2000; Heremans, 2002; Kojima, 2003; Zalzman, 2003; Zhang, 2001). Consistent with its tissue and cell-specific expression pattern, targeted disruption of the pax4 gene in mice results in the absence of mature pancreatic β- and δ-cells with a commensurate increase in the glucagon-producing α-cells (Sosa-Pineda, 1997; Wang, 2004). This increase was recently attributed to the α-cell specific transcription factor Arx that is repressed by Pax4 during development (Collombat, 2003). Normally, the earliest insulin-producing precursor cells are detected at E8.5-9 (Gittes and Rutter, 1992). Similarly, in mouse mutant Pax4 embryos, insulin-staining cells are apparent at this stage indicating that Pax4 expression is not mandatory for the generation of β-cell precursors. However, the onset of Pax4 gene expression in the pancreas around E10.5 and the absence of mature β cells in islets of Pax4 mutant newborn mice strongly suggest a critical role of this factor in the proliferation and/or survival of these early committed insulin-producing cells (Sosa-Pineda, 1997). Consistent with this hypothesis, the peak of Pax4 expression in mice was recently shown to reside between E13.5 and E15.5, a period coinciding with differentiation of β-cells, the so-called secondary transition (Wang, 2004).

In contrast to primary β-cells, elevated expression levels of Pax4 are found in human insulinomas. The same study reported the presence of a novel spliced variant of Pax4 which lacks the carboxy-terminal end of the protein involved in mediating repression of gene transcription (Miyamoto, 2001). It is presently unclear whether or not Pax4 expression is directly link to the proliferative phenotype of the insulinoma. However, ablation of the repressor domain may alleviate protein-protein interactions with potential negative regulators leading to the constitutive activation of Pax4 and ultimately cell replication. Consistent with a role of Pax4 in cell proliferation, an oncogenic function has been attributed to other closely related pax genes, e.g., the involvement of both Pax3 and Pax7 in the genesis of alveolar rhabdomyosarcoma, the responsibility of aberrant Pax5 expression for the formation of medulloblastoma and the dependence of ovarian and bladder cancer cell lines on Pax2 for survival (Davis, 1994; Galili, 1993; Kozmik, 1995; Muratovska, 2003). Interestingly suppression of Pax2 or Pax7 in tumour cell lines resulted in programmed cell death or apoptosis (Margue, 2000; Muratovska, 2003). It is noteworthy that regulation of β-cell mass during the neonatal period also implicates apoptosis (Bonner-Weir, 2000). Pax4 has indeed been shown to be down regulated in islets from newborn mice as compared to embryos (Sosa-Pineda, 1997). The role of Pax4 in determining the choice between proliferation and apoptosis is thus possible but has not yet been demonstrated. Taken together, these studies clearly demonstrate the critical role of Pax family members in cell growth and survival. Activin A, a member of the transforming growth factor β family, has independently been shown to induce Pax4 gene expression in pancreatic β cell lines (Ueda, 1996) and to stimulate growth and differentiation of human foetal pancreatic cells in combination with betacellulin (Demeterco, 2000). It will be of interest to determine whether glucose and/or the gut hormone glucagon-like peptide 1 (GLP-1) and its more stable analogue exendin-4, which also stimulate both β-cell replication and neogenesis, can modulate Pax4 gene expression (Drucker, 2001; Paris, 2003; Xu, 1999). It is noteworthy that the action of GLP-1 may be mediated by the activation of betacellulin (Buteau, 2003).

To understand the mechanisms that control the expression of Pax4 and thereby elucidate its impact on endocrine cell type determination, several studies have mapped and characterized the regulatory regions of both the human and mouse pax4 gene (Brink, 2001; Smith, 2000; Xu and Murphy, 2000). One of the most striking aspects of the pax4 gene is its capacity for auto-repression. If Pax4 functions to maintain and proliferate either β or δ-cell lineage during pancreas development, then persistent expression may be detrimental due to sustained cell division. Auto-repression will however terminate expression of Pax4 and allow expanded cells to proceed towards either a β- or δ-cell phenotype. In contrast, Pax4 expression was shown to be dependent on the concerted action of the transcription factors Pan1, Beta2/NeuroD, HNF-1α, HNF-4α and Pdx1 interacting with the promoter. A further increase in transcription was observed when Beta2/NeuroD was substituted by the early pancreatic committing transcription factor Ngn3 (Smith, 2004; Smith, 2000). The action of Beta2/NeuroD, HNF-1α, HNF-4α and Pdx1 in Pax4 gene expression is interesting given the role of these transcription factors in the development of type 2 diabetes. Thus, Pax4 may be a downstream target of these MODY-related genes. Consistent with this hypothesis, mice overexpressing a dominant negative form of HNF-1α (SM6) have morphologically normal islets at birth but gradually lose β-cell mass and developed diabetes within 6 weeks (Hagenfeldt-Johansson, 2001). In another transgenic mouse model in which a human HNF-1α mutation was targeted to the β-cells, there was already a decreased β-cell mass at birth and the adult animals displayed reduced β-cell replication (Yamagata, 2002). It is tempting to speculate that deregulation of Pax4 expression due to the absence of a functional HNF-1α effects the formation and expansion of new β-cells. The initial β-cell mass observed at birth would potentially be generated during development due to the presence of Ngn3, which could compensate for the lack of HNF-1α. Interestingly, no apparent alterations in Pax4 mRNA levels were detected in a transgenic mouse harbouring the targeted null mutation of HNF-1α (Shih, 2001). However, these studies were conducted in newborn mice, which express only low levels of Pax4 (Brink, 2001; Sosa-Pineda, 1997). Studies in mice carrying targeted mutations in MODY-genes have indicated altered expression of genes involved in glucose sensing. These alterations may underlie the insulin secretory defects observed in patients, although impaired β-cell development and proliferation could also contribute to the eventual β-cell deficiency.

The prior art, as shown above has suggested that Pax4 may be involved in differentiation of pancreatic cells during embryogenesis. Dor (2004) loc. cit. has also documented that no new islets are formed during adult life, and concludes that pre-existing β-cells are the major source for new β-cells during adult life, as well as during regeneration from (partial) pancreatectomy.

WO 98/29566 provides for a method for testing the differentiation status in/of pancreatic cells in a mammal and discloses various wild-type alleles of Pax4 (human and mouse). Pax4 is taught as a molecule involved in β-cell differentiation during embryogenesis and as an expression factor for the promotion of genes specific for insulin-producing β-cells.

Similarly, Blysczuk (2003) teaches that Pax4 expression promotes differentiation of embryonic stem cells to insulin-producing cells. Kahan (2003) uses Pax4 expression as a marker for pancreatic differentiation. Wang (2004) loc. cit., teaches that Pax4 is expressed in differentiating endocrine cells and proposes that Pax4 and NKx2.2 are two key components for initating pancreatic β-cells differentiation.

Treatment of diabetes and other pancreatic disorders is still a challenging task. Although intensive exogenous insulin therapy can approach the physiological control of blood glucose, and delay or prevent the onset of chronic complications, such treatment remains cumbersome. It is also associated with increased risk for hypoglycaemia with brain damage. The ultimate goal is treating Type 1 diabetes by the replacement of destroyed β-cells by insulin-producing cells capable of restoring glucose homeostasis in the organism.

The concept of transplantation, first attempted in the seventies, recently gained attention with the advent of the Edmonton protocol in which patients were transplanted with human islets of Langerhans isolated from brain-dead subjects. This approach has been improved by the introduction a novel immunosuppressive therapy less aggressive to the transplanted tissue (Shapiro, 2000). The largest most recent update on the clinical outcomes of the Edmonton experience was published in 2002. The authors reported 54 islet infusions in 30 patients with type 1 diabetes and provided a detailed analysis on 17 patients all of whom became insulin-independent (Ryan, 2002). However, a significant problem in human islet allotransplantation is the requirement of two to three cadaver donors to treat a single patient with diabetes (Ryan, 2001). Thus, the dramatic shortage of islets has been a barrier to the use of islet transplantation on a larger scale.

Animal donors such as pigs could provide an alternative supply of pancreas for islet isolation and transplantation. Pigs are ideal sources of xenotransplants because they are available in large numbers and because their organs are similar in size and nature to those of humans. However, pigs contain several copies of porcine endogenous retroviruses (PERV) which have been shown to infect human cells lines in vitro (Van der Laan; 2000). Thus the promise of xenotransplantation is offset by possible public health risk of a cross species infection.

The pressure to create an adequate supply of islets has led to extensive research into the potential use of islet surrogates. This include establishment of human pancreatic β-cell lines. However, research completed to date has demonstrated that human islets (Maedler, 2001) and β-cells are particularly susceptible to apoptosis (cell death) following their purification and subsequent maintenance in culture. Furthermore, conditional immortalisation of human β-cells using lentivirus technology has failed to yield stable and differentiated human cell lines. An alternative approach to generate insulin-producing cells has been the use of embryonic or adult stem cells (Weir, 2004; Street, 2004). These offer several theoretical advantages including unlimited supply, multipotency, and the possibility of being non-immunogenic. Although some progress has been achieved in generating insulin-producing cells, problems in directing homogenous differentiation have curtailed advances.

### SUMMARY OF THE INVENTION

Accordingly, the problem of the present invention is the provision of islet material to be used in medical settings for the prevention, amelioration and/or treatment of pancreatic disorders, in particular in the treatment of diabetes. The problem is solved by the provision of the embodiments of the invention as characterized in the claims and as described herein.

Accordingly, the present invention relates to an in vitro method for the generation and isolation of pancreatic β-cells, comprising the steps of:
(a) providing an adult cell derived from mammalian pancreatic islets or an explant culture of adult pancreatic islets with functional, wild-type Pax4; and
(b) detecting and isolating, from said adult cell or explant culture, β-cells that proliferate in response to the contact with (functional, wild-type) Pax4.

The above described “providing an adult cell derived from mammalian pancreatic islets or an explant culture of adult pancreatic islets with functional, wild-type Pax4” is not limited to the contact of a functional wild-type Pax4 to a single cell but also comprises the contact of whole islets which may, inter alia, be transplanted.

The “detection and isolation, from said adult cell or explant culture, β-cells that proliferate in response to the contact with (functional, wild-type) Pax4” as carried out in the method of the invention may comprise the visualization of proliferative β-cells by methods which comprise microscopical means. Proliferative β-cells may also be detected by methods which comprise cell counts. Corresponding examples and further embodiments are given in the experimental part of this invention. Said “isolation” does not always comprise the physical separation from the originally used islet (islet cells) as employed in step (a) of the method of the present invention.

The term “adult cell” relates to a cell which is not in an embryonic or foetal stage and, most preferably is not in a post-natal stage.

The term “functional, wild-type Pax4” relates to Pax4 molecules which do not comprise a mutation, ion particular no mutation which leads to a disorder or a disease, like diabetes. Such wild-type Pax4 molecules are known in the art and, inter alia, described herein in SEQ ID NOS: 1-6. Said “functional, wild-type Pax4” also comprises, however, genetic variants as well as allelic variants of the wild-type Pax4 molecules described herein. As documented in the appended examples (employing non functional or detrimental mutants), the person skilled in the art is readily in a position to deduce whether a given Pax4 molecule is a “functional, wild-type molecule”. Corresponding assays are shown in the appended examples.

In a particular embodiment of the method of the invention, the functional wild-type Pax4 is administered to the pancreatic cell, explant culture or isolated islet in form of a nucleic acid molecule. Yet, it is also envisaged that said functional wild-type Pax4 is administered to said cell, explant culture or isolated islet in form of a Pax4 gene expression product or a functional fragment thereof. Said Pax4 gene expression product may be an mRNA or a protein.

Most particularly, when said Pax4 is to be administered to the Pax4 to said cell, explant culture or isolated islet in form of a nucleic acid molecule, it is administered in form of a nucleic acid molecule comprised in a vector, whereby said vector is most particularly a viral vector. Said viral vector may, inter alia, be selected from the group from the group consisting of a retroviral vector, a lentiviral vector and an adenoviral vector. Also comprised is the use of vial particles, (like adenovial particles) or empty (adenoviral) capsids in the transfer of functional, wild-type Pax4 as desired herein. Corresponding virus-mediated co-internalization processes are known in the art and described for example in U.S. Pat. No. 5,928,944. Further envisaged are AAV, Herpex simplex virus, pox virus, measles virus, vaccinia virus or Semliki forest virus. Also “bacterial shuttles” are envisaged in accordance with this invention. Non-limiting examples of such shuttles correspondingly engineered Salmonella typhimutium and Listeria monocytogenes.

Neither the uses nor the methods described herein are limited to the vectors comprising a nucleic acid molecule encoding wild-type Pax4. It is also envisaged that other means for gene transfer, for example device-mediated or chemically mediated gene delivery, like gene gun methods, ultrasound or lipofections are employed. Also “naked” wild-type encoding Pax4 molecules may be employed in accordance with the methods and uses of this invention. Such methods and uses comprise, inter alia, the use of plasmid DNA or RNA-transfer methods known in the art. Further methods to be employed comprise polymer-based gene delivery systems, like, e.g., polyethylenimines, histidin/lysine polymers (HK) and the like.

In a particular embodiment of the invention, the Pax4 is employed in form of a nucleic acid molecule comprised in a viral vector, particularly in an adenovial vector. The adenovial vectors presently used in gene therapy protocols lack most of the E1 region which renders the virus replication deficient. Most of the adenoviral vectors use din medical settings are also E3 deleted. The feasibility of gene transfer using these vectors have successfully shown in a variety of tissues in vivo and in vitro; U.S. Pat. No. 6,099,831 or U.S. Pat. No. 6,013,638. As documented in the appended examples, adenoviral vector which may be employed in context of this invention are AdCMV or pHVAd2 as described in detail herein. Also RNA-inhibiting/RNA-interference approaches have been carried out in order to document the surprising and direct contribution of Pax4 on the survival of insulin-producing cells.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D: Activin A and betacellulin increase Pax4 mRNA levels in a dose dependent manner in mature rat pancreatic islets. (FIG. 1A) Low levels of Pax4 are expressed in adult rat islets. Quantitative real time RT-PCR using RNA purified from freshly isolated rat islets and INS-1E cells. Data are presented as percent of Pax4 mRNA levels relative to INS-1E. Each value (n=6 independent experiments) represents the mean±SE. Quantitative real-time RT-PCR analysis of PAX4 and insulin mRNA steady state levels in isolated rat islets treated with increasing doses of activin A (FIG. 1B), betacellulin (FIG. 1C) or TGF-β1 (FIG. 1D) for 24 h. Total RNA from 50 islets was extracted and reverse transcribed into cDNA as described in “experimental procedures”. For each sample (20 ng), three distinct amplifications were performed in parallel and mean values for PAX4 and insulin were normalized to the mean value of the reference housekeeping gene cyclophilin. Results are expressed as the relative fold increase of the stimulated over the control group. Each value represents the mean±SE of at least 4 independent experiments. *, P<0.05.

FIGS. 2A-B: Activin A and betacellulin increase β-cell proliferation in mature rat islets. (FIG. 2A) Immunofluorescent detection of BrdU (first row), insulin (second row) and merged image including nuclear DAPI staining (third row) in dispersed islet cells 48 h after incubation with the indicated growth factors. The proliferation was measured using the BrdU incorporation assay as in FIGS. 4A-C. Magnification ×400. (FIG. 2B) β-cells immunostained for both insulin and BrdU were counted and results are expressed as a percentage of BrdU/insulin positive cells over the total number of insulin positive cells. Calculated values are depicted below the graph. Data represent the mean±SE of 4 independent experiments, comprising more than 900 cells per condition. Statistical significance was tested between control islets and islets incubated with the various growth factors by Student's t test. **, P<0.01

FIGS. 3A-B: Pax4 is overexpressed in isolated rat pancreatic islets after infection with the recombinant adenovirus AdCMVPax4IRESGFP. (FIG. 3A) Immunofluorescent detection of EGFP (first row), insulin (second row) as well as nuclei staining with DAPI (third row) in dispersed islet cells 48 h after infection with the indicated doses of adenovirus. Pax4 overexpression is identified via the reporter co-translated EGFP in insulin positive cells. Of note, the viral infection does not induce nuclear damage as assessed by DAPI staining. Magnification ×400. (FIG. 3B) EMSA using a radiolabeled G3 element of the glucagon gene promoter and full-length mouse Pax4, produced in vitro with the coupled TNT system (lanes 1, 2, 3) as well as nuclear protein extracts (6 μg) from infected rat islets (lanes 4 to 8). Infection for 48 h with the indicated amounts of the adenovirus increased Pax4 DNA binding activity to the G3 element in a dose-dependent manner (lanes 5, 6, 7). The asterisk * delineates the formation of a supershift complex due to the addition of anti-Pax4 serum (lanes 2 and 8).

FIGS. 4A-C: Overexpression of Pax4 induces β-cell proliferation in AdCMVPaxIRESGFP-transduced rat islets. (FIG. 4A) Immunocytochemical detection of BrdU (first row) and insulin (second row) in dispersed islet cells 48 h after infection with various recombinant adenoviruses (2.4×10⁷ pfu/ml). The merged image also includes DAPI staining (third row) to highlight nuclei. Cells were labeled with 10 μM BrdU for the last seven h of incubation and proliferation was visualized using the BrdU incorporation assay. Magnification ×400. (FIG. 4B) Merged image of BrdU (green in colour print), insulin (red in colour print) and DAPI (blue in colour print) of intact islets infected with either AdCaLacZ or AdCMVPax4IRESGFP. (FIG. 4C) β-cells immunostained for both insulin and BrdU were counted under a fluorescent microscope. Results are expressed as a percentage of BrdU/insulin positive cells over the total amount of insulin positive cells and values are depicted below the graph. Data show the mean±SE of 4 independent experiments, each representing more than 1000 cells per condition. Statistical significance was tested between LacZ- and the transcription factors Pax4-, Pax6- and neurogenin3 (Ngn3)-overexpressing islets by Student's t test. **, P<0.01

FIGS. 5A-C: Time-dependent gene expression profiling of Pax4 overexpressing rat islets. (FIG. 5A) EMSA using nuclear protein extracts (6 μg) from AdCMVPax4IRESGFP transduced rat islets cultured in RPMI 1640 medium over a period of 6 days. Pax4 DNA binding activity to the G3 element is maximal one-day post infection. The asterisk * represents the supershifted complex in the presence of anti-Pax4 serum. (FIGS. 5B-C) Quantitative real-time RT-PCR analysis performed on RNA isolated from AdCaLacZ (LacZ)-() and AdCMVPaxIRESGFP (PAX4)-(▪) infected islets (2.4×10⁷ pfu/ml). Transcript levels were grouped into 4 categories; proliferative genes comprising c-myc and Id2; apoptotic genes composed of Bcl-xL, Bcl-2 and caspase-3; the transcription factor Pdx-1 and finally endocrine hormone genes comprising insulin, glucagon and somatostatin. Expression patterns were measured over a period of 6 days. Each value represents mean±SE of 3 independent experiments. Statistical significance was tested between LacZ and PAX4 infected islets by unpaired Student's t test. *, P<0.05 **, P<0.01.

FIGS. 6A-C: Analysis of the expression and function of Pax4 wt and its mutant R129W. (FIG. 6A) Immunofluorescent detection of the myc-tagged Pax4 or synaptotagmin VII proteins (first row) and nuclei staining with DAPI (second row) in INS1E cells 48 h after transfection with the indicated constructs. The transcription factor Pax4 and synaptotagmin VII were detected via the myc epitope in the nuclei and cytoplasm of INS1E cells, respectively; the R129W mutation does not affect Pax4 nuclear localisation. Magnification ×400. (FIG. 6B) EMSA using a radiolabeled G3 element of the glucagon gene promoter and the recombinant proteins Pax4-myc wt (lanes 1 and 2), and Pax4-myc R129W (lanes 3 and 4). An equal amount of protein, produced in vitro with the coupled TNT system, was applied in each lane. Wild-type Pax4 bound to the G3 element (lane 1) whereas the R129W mutation strongly decreased the DNA binding activity to the probe (lane 3). The asterisk * delineates the formation of a supershift complex due to the addition of anti-myc epitope antibody (lanes 2 and 4). (FIG. 6C) Effects of Pax4-myc wt (▪) and its mutant R129W (□) on the human c-myc promoter and murine Bcl-xL promoter. Transient cotransfection studies using BHK-21 cells were performed with increasing amounts of wt and mutant Pax4. The telomerase promoter construct (▴) was used as an internal control in Pax4-myc wt-transfected BHK-21 cells. Data are presented as fold induction of basal luciferase activity and expressed as the mean±SEM of 4-5 independent experiments. Pax4-myc transcriptionally activated the c-myc and Bcl-xL promoters whereas the mutant showed a significant reduction of transcriptional activation indicated by * (P<0.05).

FIGS. 7A-B: Effects of Pax4 overexpression on insulin secretion and glucose oxidation in isolated rat islets. (FIG. 7A) Glucose-induced insulin secretion was inhibited by AdCMVPax4IRESGFP in a dose dependent manner. Two days after infection, islet hormone secretion was assayed as described in experimental procedures. Data are expressed as the mean±SEM of 4 independent experiments. **=P<0.01 as analysed unpaired Student's t-test. (FIG. 7B) Two days post infection with 2.4×10⁷ pfu/ml of indicated adenoviruses, islet CO₂ generation was measured in the presence of 2.5 mM or 16.7 mM glucose to assess glucose oxidation rate as described in the experimental procedures. Data represent the mean±SEM of 5 independent experiments.

FIGS. 8A-C: Both cellular ATP and mitochondrial calcium levels are increased in AdCMVPax4IRESGFP-infected islets. (FIG. 8A) Total cellular ATP levels were measured in islets overexpressing either β-galactosidase or PAX4 (2.4×10⁷ pfu/ml) and maintained in 1 mM glucose for 10 min. Results represent the means±SE. **P<0.01. (FIG. 8B) Cytosolic ATP production in response to 2.5 or 16.5 mM glucose was also determined over a period of 20 min using the ATP-sensitive bioluminescence probe luciferase. Luminescence was recorded in a FLUOstar Optima apparatus. Islets were equilibrated in KRBH buffer for 30 mM prior to initiation of recording. Glucose and azide were added at indicated times (arrows). Results are the mean±SE of at least 5 experiments performed in duplicates. (FIG. 8C) Mitochondrial calcium was monitored in LacZ or PAX4 overexpressing islets using β-cell specific/mitochondrial-targeted aeqourin. Islets infected with rAdRIPmAQ (4.8×10⁸ pfu/ml) and either AdCaLacZ or AdCMVPax4IRESGFP (2.4×10⁷ pfu/ml) were cultured for 65 h prior to experimentation. After the establishment of baseline luminescence (30 min; LacZ=210±49 nM and Pax4=387±46 nM), islets were superfused for 5 min in basal conditions (0 glucose) before stimulation with glucose (16.7 mM), and then KCl (60 mM), for 5 min intervals each, as shown (inset). The induced increases in [Ca²⁺]ₘ were evaluated on the basis of peak height and area under the peak (AUP). The elapsed time from the addition of glucose to the start of the calcium response (reaction time), and basal calcium (at 200-300 s) was also compared. Results for Pax4 overexpressing islets are expressed as a percentage of those for LacZ controls (100%). Each value represents the mean±SEM of a minimum of 6 separate experiments. *=p<0.02, **=p<0.055.

FIG. 9: Proposed model of Pax4-induced β-cell proliferation. Based on these results, the inventors propose that mitogens such as activin A and betacellulin activate Pax4, which will stimulate c-myc and Bcl-xL gene transcription. c-myc will then promote Id2 gene expression and activate the cell cycle replication program. Bcl-xL increased expression will promote proliferation by preventing mitochondria from initiating the apoptotic program. However, cells become refractory to glucose-evoked insulin secretion due to altered ATP production and handling as well as calcium homeostasis.

FIG. 10: Insulin and glucagon protein contents in Pax4 overexpressing islets. Total insulin and glucagon protein contents were quantified by radioimmunoassay 48 h post infection and results were expressed as ng of protein per islet. Data show the mean±SEM of 3 independent experiments.

FIGS. 11A-B: Mitogens as well as Pax4 overexpression promotes human islet β-cell replication (FIG. 11A) Quantitative real-time RT-PCR analysis of PAX4 mRNA steady state levels in isolated human islets treated with 11 or 25 mM glucose for 24 and 48 hours as well as with increasing doses of activin A, betacellulin or TGF-β1 for 24 h. Total RNA from 100 islets was extracted and reverse transcribed into cDNA. For each sample (20 ng), three distinct amplifications were performed in parallel and mean values for PAX4 were normalized to the mean value of the reference housekeeping gene cyclophilin. Results are expressed as the relative fold increase of the stimulated over the 5.5 mM glucose control group. (FIG. 11B) Immunocytochemical detection of BrdU (first row) in dispersed human islet cells 48 h after infection with various recombinant adenoviruses. The merged image includes DAPI staining (second row) to highlight nuclei. Magnification ×400.

FIGS. 12A-B: Construction and analysis of the expression of mouse Pax4-myc wild-type (WT) and its mutant R129W. (FIG. 12A) The full-length mouse Pax4 cDNA (represented schematically) was amplified by polymerase chain reaction (PCR) and subcloned into the expression vector pcDNA3.1/e-myc/6-His (Invitrogen). The myc-tagged was incorporated in order to detect Pax4 by immunofluorescence since available antibodies fail to recognise the transcription factor. The Pax4-myc wild-type (WT) was subjected to mutagenesis to substitute an arginine to a tryptophan at position 129 (Pax4-myc R129W). This mutation, located in the paired DNA binding domain, corresponds to the human Pax4 mutation R121W which has been associated with late onset type 2 diabetes. (FIG. 12B) EMSA was subsequently performed using recombinant Pax4-myc WT or R129W proteins generated in vitro in the presence of the glucagon promoter element G3, a bona fide Pax4 binding site. Wild-type Pax4 bound to the G3 element (lane 1) whereas the R129W mutation strongly decreased the DNA binding activity to the probe (lane 3). The asterisk * delineates the formation of a supershift complex due to the addition of anti-myc epitope antibody (lanes 2 and 4). Lane 5, c-myc epitope antibody.

FIGS. 13A-B: Expression of Pax4 induces human islet cell proliferation in an adenoviral-mediated inducible expression system. (FIG. 13A) Immunofluorescent detection of the myc-tagged Pax4 protein (Pax4 in first row), nuclei staining with DAPI (DAPI in second row) and merged image in dispersed human islet cells 48 hrs after infection with the recombinant adenovirus Ad-mPax4-myc WT (2.4×10⁷ pfu/ml) and the adenoviral construct harbouring the tetracycline transcriptional activator Ad-X Tet-On (1.2×10⁷ pfu/ml). Infected cells were cultured in the absence or presence of the inducer doxycycline (0.5 μg/ml). The transcription factor Pax4 was detected, via the c-myc epitope, in the nuclei of approximately 70% of human islet cells cultured in the presence of doxycycline, while no basal induction of Pax4 was observed in the absence of doxycycline. Nuclear fragmentation was absent in transduced cells indicating that viral infection did not affect cell viability or apoptosis. Magnification ×400. (FIG. 13B) Immunocytochemical detection of BrdU incorporation (first row), nuclei staining (second row) and merged image in human islet cells 48 hrs after infection with the adenoviral-mediated inducible expression system (see above) and cultured in the absence or presence of doxycycline (0.5 μg/ml). Cells were labeled with 10 μM BrdU for 48 hrs of incubation and proliferation was visualized using the BrdU incorporation assay. Forced expression of Pax4 induced proliferation in human islet cells. The merged image confirms the nuclear incorporation of BrdU. Magnification ×400.

FIGS. 14A-B: Induction of mutant Pax4 R129W had no effect on human islet cell proliferation. (FIG. 14A) Immunofluorescent detection of the c-myc-tagged mutant Pax4 R129W protein (Pax4 first row), nuclear DAPI staining (second row) and merged image in dispersed human islet cells 48 hrs after infection with the recombinant adenovirus Ad-mPax4-myc R129W (2.4×10⁷ pfu/ml) and the adenovirus Ad-X Tet-On (1.2×10⁷ pfu/ml). Infected cells were cultured in the absence or presence of doxycycline (0.5 μg/ml). The R129W mutation does not affect Pax4 nuclear localisation in infected human islet cells cultured in the presence of doxycycline. Magnification ×400. (FIG. 14B) Immunocytochemical detection of BrdU incorporation (green), nuclei staining (DAPI in blue) and merged image in human islet cells 48 hrs after infection with the adenoviral-mediated inducible expression system (see above) and cultured in the absence or presence of doxycycline (0.5 μg/ml). The proliferation was measured using the BrdU incorporation assay as in FIG. 2B. Magnification ×400.

FIG. 15: Doxycycline-stimulated Pax4 expression completely protects human islets from cytokine-induced apoptosis. Human islets were infected with the recombinant adenovirus Ad-mPax4-myc WT and the adenovirus Ad-X Tet-On and cultured for 24 h in the absence or presence of the indicated concentrations of doxycycline. Islets were subsequently treated for 24 h with IFN-γ, IL-1β and TNF-α (2 ng/ml each) to promote apoptosis. (A) Immunocytochemical detection of apoptotic cells (first row) using TUNEL assay, nuclei staining (second row) and merged image in human islet cells 48 hrs after infection with the adenoviral-mediated inducible expression system and cultured in the absence or presence of doxycycline (0.5 μg/ml) in the presence of cytokines. The merged image confirms the nuclear incorporation of fluorescein in apoptotic cells. Magnification ×400. (B) Cell death was measured by the TUNEL assay and results were expressed as a percentage of fluorescein-labeled nuclei (TUNEL-positive cells) over the total amount of islet cells (nuclei staining by DAPI). Data show the mean±SEM of four independent experiments, each representing more than 700 cells per condition *, p<0.05 and ** p<0.01.

FIG. 16: Induction of mutant Pax4 R129W partially protects human islets from cytokine-induced apoptosis. Human islets were infected with the recombinant adenovirus Ad-mPax4-myc R129W and the adenovirus Ad-X Tet-On as described in Materials and Methods and cultured for 24 h in the absence or presence of the indicated concentrations of doxycycline. Islets were subsequently treated for 24 h with IFN-γ, IL-1β and TNF-α (2 ng/ml each) to promote apoptosis. (A) Immunocytochemical detection of apoptotic cells (first row) using TUNEL assay, nuclei staining (second row) and merged image in human islet cells 48 hrs after infection with the adenoviral-mediated inducible expression system and cultured in the absence or presence of doxycycline (0.5 μg/ml) in the presence of cytokines. The merged image confirms the nuclear incorporation of fluorescein in apoptotic cells. Magnification ×400. (B) Cell death was measured by the TUNEL assay and results were expressed as a percentage of fluorescein-labeled nuclei (TUNEL-positive cells) over the total amount of islet cells (nuclei staining by DAPI). Data show the mean±SEM of four independent experiments, each representing more than 700 cells per condition *, p<0.05 and ** p<0.01.

FIG. 17: Activin A and betacellulin increase endogenous Pax4 mRNA levels in human islets. Pax4 mRNA levels in human islets treated with 0.5 nM of activin A, betacellulin or TGF-β1 in the presence of 5.5 or 11 mM glucose for 24 hours as indicated in the figure. Relative Pax4 mRNA abundance levels were measured by quantitative real-time RT-PCR and normalized to the transcript cyclophilin. Data represent the mean of 3-5 independent experiments.

FIG. 18: Development of a RNA interference strategy (RNAi) and its impact an the expression of endogenous rat Pax4, Pdx1 and Bcl-xL gene expression in INS-1E cells. (A) Schematic representation of Pax4 protein structure depicting the two highly conserved DNA binding domains (paired and homeo domains) as well as a repressor/activator domain. The three Pax4 siRNA structures (two targeted to the paired domain, siPD21 and siPD29 and one to the homeo domain, siHD21) are represented with bars. Quantitative real-time RT-PCR analysis of rat Pax4 (B), PDX1 (C) and Bcl-xL (D) mRNA steady state levels in INS-1E cells co-transfected with an empty pDLDU6 vector (U6, control) or with a pDLDU6 vector containing the different Pax4 siRNA (siPD21, siPD29 or siHD21) along with an expression vector for GFP. Seventy two hours post-transfection, GFP⁻ (white bars) and GFP⁺ (black bars) cells were purified by FACS, RNA was extracted and Pax4, PDX1 as well as Bcl-xL mRNA steady state levels were evaluated by real time RT-PCR and normalized to the transcript cyclophilin. Results are the mean of 2 independent experiments performed in triplicates and are expressed as fold changes as compared to the GFP⁻ cells.

FIG. 19: INS-1E cells transfected with either siPD21, siPD29 or siHD21 are more sensitive to cytokine-induced cell death. INS-1E cells were co-transfected with the control vector (U6) or with the different Pax4 siRNA (siPD21, siPD29 or siHD21) along with a phogrin-GFP plasmid and were cultured for 48 hours. INS-1E cells were subsequently treated for 24 h with IFN-γ, IL-1β and TNF-α (1 ng/ml each) to promote apoptosis. Immunocytochemical detection of apoptotic cells using TUNEL assay and phogrin-GFP expressing cells and merged image with nuclei staining. The merged image confirms the nuclear incorporation of rhodamin in apoptotic cells. Magnification ×400.

### DETAILED DESCRIPTION OF THE INVENTION

In accordance with the present invention it has been surprisingly found that Pax4, in contrast to the teaching of the prior art, is not only involved in β-cell differentiation during regeneration of β-cells or during embryogenesis, but that it can also successfully be used to initiate cell proliferation of pre-existing, particularly adult pancreatic β-cells. The advantage of the method of the present invention is the provision of larger quantities of pancreatic β-cells which may be used in particular transplantation approaches for the medical intervention in pancreatic diseases, in particular in diabetes. Results presented in the experimental part of this invention show that Pax4 promotes β-cell proliferation and is capable of preventing cell death, in particular by increased expression of the Bcl-xL gene. This documents that terminally differentiated adult β-cells retain a proliferative capacity and can be exploited as an alternative source for cell- or tissue-based therapy.

Polymorphisms in the pax4 gene have been associated with Type 1 diabetes while point mutations have been linked to Type 2 diabetes, implicating Pax4 in mature β-cell function and/or regeneration. As documented in the appended examples, induction of endogenous Pax4 gene expression coincides with β-cell proliferation induced by the mitogens activin A and betacellulin in adult rat islets. Consistent with a proliferative role of Pax4, we also demonstrated using recombinant adenoviruses, that rat and human β-cells overexpressing Pax4 displayed greater replication rates as compared to control-infected islets. In context of the present invention the impact of mitogens on endogenous Pax4 expression both at the transcript and protein level in human islets was evaluated. Furthermore, in order to evaluate the direct contribution of Pax4 on β-cell plasticity, a RNA interference (RNAi) strategy to suppress Pax4 activation in response to mitogens; see also Example 11.

Immunofluorescence studies were performed on partially trypsinized human islets for the transcription factors Pax4, Pdx1, Nkx6.1, Isl1, Ngn3 and insulin. Isolated human islets were exposed to either 5.5 or 11 mM glucose for 24 and 48 hours. Islets were also treated with 0.5 nM of activin A, betacellulin or TGF-β1 for 24 hours. Steady state mRNA levels for Pax4 were quantified by quantitative real-time RT-PCR and normalized to cyclophilin. A 21-nucleotide Pax4 hairpin RNA structures (siPax4) was cloned into the pDLDU6 vector and transfected into the rat insulinoma cell line INS1E along with GFP using lipofectamine. Subsequent to cell sorting using GFP (72 hours post-transfection), the effects of RNAi on endogenous Pax4 transcript levels were quantified by real time RT-PCR.

A comparative profile of transcription factors in human islets was performed initially to establish relative expression levels of Pax4. Low but consistent levels of Pax4 mRNA and protein were detected in isolated human islets as compared to Nkx6.1, Pdx1 and Isl1. In contrast, Ngn3 was undetectable. In parallel, it was found that exposure to 11 mM glucose for 48 hours resulted in a 3.6-fold increase in Pax4 mRNA levels as compared to control 5.5 mM glucose. Treatment with either 0.5 nM activin A or betacellulin for 24 hours resulted in a 3.5- and 8 fold increase in Pax4 transcript, respectively. In contrast, TGF-β1 was ineffective. Accordingly, Pax4 activity is regulated by physiological stimuli in human islets. The insulinoma INS1E cells expressed high levels of Pax4 mRNA. Pax4 steady state mRNA levels were lowered by 80% in INSIE cells co-expressing GFP and an interfering/inhibiting RNA, namely siPax4 (see also Example 11). Repression was specific since mRNA levels for PDX1 and insulin remained constant.

Therefore, Pax4 is induced by mitogens known to promote pancreatic islet cell proliferation.

As will be detailed herein below, in particular ex vivo gene delivery of Pax4 into terminally differentiated adult β-cells can be employed to expand β-cells in culture and to reduce the number of human islets required for successful transplantation.

### I. Pax4 Proteins and Nucleic Acids

The functional, wild-type Pax4 to be particularly used in context of this invention is the wild-type Pax4 of mouse, rat or human. Most particularly, the functional, wild-type Pax4 to be employed is human Pax4. Corresponding molecules (i.e., nucleotide sequences and amino acid sequences) are known in the art and also shown herein below. Accordingly, in a particular embodiment of the invention, the functional, wild-type Pax4 to be employed in the method and uses provided herein is encoded by
(a) a nucleic acid molecule comprising a nucleic acid molecule encoding the polypeptide having the amino acid sequence as shown in SEQ ID NO: 2 (rat), 4 (mouse), or 6 (human);
(b) a nucleic acid molecule comprising a nucleic acid molecule having the DNA sequence as shown in SEQ ID NO: 1 (rat), 3 (mouse), or 5 (human);
(c) a nucleic acid molecule hybridizing to the complementary strand of nucleic acid molecules of (a) or (b) and encoding a functional wild-type Pax4; and
(d) a nucleic acid molecule being degenerate as a result of the genetic code to the nucleotide sequence of the nucleic acid molecule as defined in (c).

In accordance with this invention, also Pax4 molecules may be employed which are highly homologous to the functional, wild-type Pax4 molecules known in the art and disclosed herein. Yet, this molecules have to be functional in the sense that these molecules do stimulate the proliferation of β-cells as documented herein and that these functional molecules do not provoke detrimental effects as shown with detrimental mutant forms presented in the experimental part of this invention. Accordingly, the invention also provides for a “read-out system” whether a give Pax4 molecule is “functional”, i.e., capable of stimulating β-cell proliferation of adult (islet) cells. Such “functional”, wild-type Pax4 molecules are known in the art, and comprise, but are not limited to the sequences provided herein or disclosed in U.S. Pat. No. 6,071,697. Further “functional, wild-type Pax4” molecules are encoded by nucleic acid molecules as provided in the GeneBank under accession numbers: (rat Pax4) NM_{—}031799 (mousePax4) NM_{—}011038 and (humanPax4): AF043978 or NM_{—}006193.

In order to determine whether a nucleic acid sequence has a certain degree of identity to the nucleic acid sequence encoding a wild-type Pax4 as defined above, the skilled person can use means and methods well-known in the art, e.g., alignments, either manually or by using computer programs such as those mentioned further down below in connection with the definition of the term “hybridization” and degrees of homology.

For example, BLAST2.0, which stands for Basic Local Alignment Search Tool (Altschul, 1997; Altschul, 1993); Altschul, 1990), can be used to search for local sequence alignments. BLAST produces alignments of both nucleotide and amino acid sequences to determine sequence similarity. Because of the local nature of the alignments, BLAST is especially useful in determining exact matches or in identifying similar sequences. The fundamental unit of BLAST algorithm output is the High-scoring Segment Pair (HSP). An HSP consists of two sequence fragments of arbitrary but equal lengths whose alignment is locally maximal and for which the alignment score meets or exceeds a threshold or cutoff score set by the user. The BLAST approach is to look for HSPs between a query sequence and a database sequence, to evaluate the statistical significance of any matches found, and to report only those matches which satisfy the user-selected threshold of significance. The parameter E establishes the statistically significant threshold for reporting database sequence matches. E is interpreted as the upper bound of the expected frequency of chance occurrence of an HSP (or set of HSPs) within the context of the entire database search. Any database sequence whose match satisfies E is reported in the program output.

Analogous computer techniques using BLAST (Altschul (1997), loc. cit.; Altschul (1993), loc. cit.; Altschul (1990), loc. cit.) are used to search for identical or related molecules in nucleotide databases such as GenBank or EMBL. This analysis is much faster than multiple membrane-based hybridizations. In addition, the sensitivity of the computer search can be modified to determine whether any particular match is categorized as exact or similar. The basis of the search is the product score which is defined as:

$$\frac{\% sequence identity \times \% maximum BLAST score}{100}$$

and it takes into account both the degree of similarity between two sequences and the length of the sequence match. For example, with a product score of 40, the match will be exact within a 1-2% error; and at 70, the match will be exact. Similar molecules are usually identified by selecting those which show product scores between 15 and 40, although lower scores may identify related molecules.

The present invention also relates to wild-type Pax4 nucleic acid molecules which hybridize to one of the above described nucleic acid molecules and which encode a functional Pax4 molecule as described herein, i.e., a Pax4 molecules which does not lead to disorders or diseases, like diabetes.

The term “hybridizes” as used in accordance with the present invention may relate to hybridization under stringent or non-stringent conditions. If not further specified, the conditions are particularly non-stringent. Said hybridization conditions may be established according to conventional protocols described, for example, in Sambrook, (2001); Ausubel, (1996), or Higgins and Hames, (1985). The setting of conditions is well within the skill of the artisan and can be determined according to protocols described in the art. Thus, the detection of only specifically hybridizing sequences will usually require stringent hybridization and washing conditions such as 0.1×SSC, 0.1% SDS at 65° C. Non-stringent hybridization conditions for the detection of homologous or not exactly complementary sequences may be set at 6×SSC, 1% SDS at 65° C. As is well known, the length of the probe and the composition of the nucleic acid to be determined constitute further parameters of the hybridization conditions. Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility. Hybridizing nucleic acid molecules also comprise fragments of the above described molecules. Such fragments may represent nucleic acid sequences which encode wild-type Pax4 which lacks the repressor region, as inter alia, encoded by the nucleic acid molecule shown in SEQ ID No. 13. Accordingly, envisaged in context of this invention is a wild-type Pax4 molecule which lacks the repressor domain. Such a wild-type Pax4 molecule may be encoded by a (human) Pax4 nucleic acid molecule as shown below (SEQ ID NO. 13) lacking the repressor domain. Such a construct may show high proliferative capacity on human islet β-cells.

Furthermore, nucleic acid molecules which hybridize with any of the aforementioned nucleic acid molecules also include complementary fragments, derivatives and allelic variants of these molecules as long as these molecules are capable of inducing β-cell proliferation of in adult pancreatic cells and do not lead to disorders like diabetes. Additionally, a hybridization complex refers to a complex between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., Cot or Rot analysis) or between one nucleic acid sequence present in solution and another nucleic acid sequence immobilized on a solid support (e.g., membranes, filters, chips, pins or glass slides to which, e.g., cells have been fixed). The terms complementary or complementarity refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between single-stranded molecules. The degree of complementarity between nucleic acid strands has significant effects on the efficiency and strength of hybridization between nucleic acid strands. This is of particular importance in amplification reactions, which depend upon binding between nucleic acids strands.

The term “hybridizing sequences” particularly refers to sequences which display a sequence identity of at least 40%, particularly at least 50%, more particularly at least 60%, even more particularly at least 70%, particularly at least 80%, more particularly at least 90%, even more particularly at least 95%, 97% or 98% and most particularly at least 99% identity with a nucleic acid sequence as described above encoding a wild-type Pax4.

In accordance with the present invention, the term “identical” or “percent identity” in the context of two or more nucleic acid or amino acid sequences, refers to two or more sequences or subsequences that are the same, or that have a specified percentage of amino acid residues or nucleotides that are the same (e.g., 70-95% identity, more particularly at least 95%, 97%, 98% or 99% identity), when compared and aligned for maximum correspondence over a window of comparison, or over a designated region as measured using a sequence comparison algorithm as known in the art, or by manual alignment and visual inspection. Sequences having, for example, 95% or greater sequence identity are considered to be substantially identical. Such a definition also applies to the complement of a test sequence. Those having skill in the art will know how to determine percent identity between/among sequences using, for example, algorithms such as those based on CLUSTALW computer program (Thompson, 1994; or FASTDB (Brutlag, 1990, as known in the art.

Recombinant vectors form important further aspects of the present invention. The term “expression vector or construct” means any type of genetic construct containing a nucleic acid coding for a gene product in which part or all of the nucleic acid encoding sequence is capable of being transcribed. Particularly useful vectors are contemplated to be those vectors in which the coding portion of the DNA segment, whether encoding a full length protein or smaller peptide, is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned”, “under control” or “under transcriptional control” means that the promoter is in the correct location and orientation in relation to the nucleic acid to control RNA polymerase initiation and expression of the gene.

The promoter may be in the form of the promoter that is naturally associated with a particular gene, as may be obtained by isolating the 5′ non-coding sequences located upstream of the coding segment or exon, for example, using recombinant cloning and/or polymerase chain reaction (PCR™) technology, in connection with the compositions disclosed herein (PCR technology is disclosed in U.S. Pat. No. 4,683,202 and U.S. Pat. No. 4,682,195, each incorporated herein by reference).

In other embodiments, it is contemplated that certain advantages will be gained by positioning the coding DNA segment under the control of a recombinant, or heterologous, promoter. As used herein, a recombinant or heterologous promoter is intended to refer to a promoter that is not normally associated with a particular gene in its natural environment. Such promoters may include promoters normally associated with other genes, and/or promoters isolated from any other bacterial, viral, eukaryotic, or mammalian cells.

Naturally, it will be important to employ a promoter that effectively directs the expression of the DNA segment in the cell type chosen for expression. The use of promoter and cell type combinations for protein expression is generally known to those of skill in the art of molecular biology, for example, see Sambrook et al. (2001), incorporated herein by reference. The promoters employed may be constitutive, or inducible, and can be used under the appropriate conditions to direct high level expression of the introduced DNA segment, such as is advantageous in the large-scale production of recombinant proteins or peptides.

A. Promoters

The promoter is required to express the transforming genetic construct to a degree sufficient to effect transformation of a target cell type amongst a population of different cell types such that the transformed target cell results in the generation of a stable human regulated secretory cell. Promoters can be classified into two groups, ubiquitous and tissue- or cell-specific. Ubiquitous promoters activate transcription in all or most tissues and cell types. Examples of ubiquitous promoters are cellular promoters like the histone promoters, promoters for many metabolic enzyme genes such as hexokinase I and glyceraldehyde-3-phosphate dehydrogenase, and many viral promoters such as the cytomegalovirus promoter (CMVp) and the Rous sarcoma virus promoter (RSVp). In certain aspects of the present invention, these promoters are appropriate for use with the immortalizing constructs described herein, as well as finding use in additional aspects of the present invention.

Tissue- or cell-specific promoters activate transcription in a restricted set of tissues or cell types or, in some cases, only in a single cell type of a particular tissue. Examples of stringent cell-specific promoters are the insulin gene promoters which are expressed in only a single cell type (pancreatic β-cells) while remaining silent in all other cell types, and the immunoglobulin gene promoters which are expressed only in cell types of the immune system.

The promoter may also be “context specific” in that it will be expressed only in the desired cell type and not in other cell types that are likely to be present in the population of target cells, e.g., it will be expressed in β-cells, but not in α- or δ-cells, when introduced into intact human islets. For example, an insulin promoter targets the expression of a linked transforming oncogene selectively to β-cells of a human islet preparation even though many other contaminating cell types exist in the preparation.

1. β-Cell-Specific Promoters

It has been documented that the two rat insulin gene promoters, RIP1 (GenBank accession number J00747) and RIP2 (GenBank accession number J00748), as well as the human insulin promoter (HIP; GenBank accession number V00565), direct stringent cell-specific expression of the insulin gene in rodent β-cell insulinoma lines (German et al., 1990), primary islet cells (Melloul et al., 1993), and in β-cells of transgenic mice (Efrat et al., 1988).

As the sequence and position of the functional promoter elements are well conserved between HIP, RIP1 and RIP2, the transcription factors that interact with these elements are likely to be conserved across species. In fact, HIP can direct cell-specific expression of linked genes in rodent β-cell lines and rat primary islets, albeit, at a somewhat lower level than that observed for RIP1 (Melloul et al., 1993). Melloul et al. (1993) demonstrated that the isolated 50-bp RIP1 FAR/FLAT minienhancer (FF), an essential promoter element for RIP1 activity, could express a linked reporter gene in both adult rat and human islet cells. Furthermore, FF activity could be substantially induced by increased concentrations of glucose in both species of adult islets. Additional results from gel-shift studies strongly suggested that the same or similar β-cell-specific transcription factor(s) from both adult rat and human islet cell nuclear extracts bound to conserved sequences contained within both the RIP1 FF and the analogous region of HIP.

2. Modified Promoters

Promoters can be modified in a number of ways to increase their transcriptional activity. Multiple copies of a given promoter can be linked in tandem, mutations which increase activity may be introduced, single or multiple copies of individual promoter elements may be attached, parts of unrelated promoters may be fused together, or some combination of all of the above can be employed to generate highly active promoters. All such methods are contemplated for use in connection with the present invention.

German et al. (1992a) mutated three nucleotides in the transcriptionally important FLAT E box of the rat insulin I gene promoter (RIP), resulting in a three- to four-fold increase in transcriptional activity of the mutated RIP compared to that of a nonmutated RIP as assayed in transiently transfected HIT cells. Also, the introduction of multiple copies of a promoter element from the E. coli tetracycline resistance operon promoter were introduced into the CMV promoter, significantly increasing the activity of this already very potent promoter (Liang et al., 1996). Additionally, part of the CMV promoter, which has high but short-lived transcriptional activity in dog myoblasts, was linked to the muscle-specific creatine kinase promoter (MCKp), which has weak but sustained expression in dog myoblasts, resulting in a hybrid promoter that sustained high-level expression for extended periods in dog myoblasts.

3. Multimerized Promoters

Several modified rat insulin promoters (modRIP) containing multimerized enhancer elements have been engineered. The currently preferred modRIP contains six multimerized repeats of a 50 base pair region of the cis acting enhancer of RIP, placed upstream of an intact copy of RIP. These novel promoters have been shown to direct expression of transgenes in stably engineered β-cell lines at levels above those attained with unmodified insulin promoters and, in some cases, approaching the levels achieved with the cytomegalovirus promoter (CMVp). CMVp is one of the strongest activating promoters known, but in a very non-tissue specific manner.

B. DNA Delivery

In certain embodiments of the invention, the nucleic acid encoding the one or more product(s) of interest may be integrated into the host cell's genome. In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. All delivery methods are contemplated for use in the context of the present invention, although certain methods are preferred, as outlined below.

1. Transfection

In order to effect expression, the construct must be delivered into a cell. As described below, the preferred mechanism for delivery is via viral infection, where the construct is encapsidated in an infectious viral particle. However, several non-viral methods for the transfer of one or more immortalizing or other expression constructs into cultured mammalian cells also are contemplated by the present invention. In one embodiment of the present invention, the expression construct may consist only of naked recombinant DNA or plasmids. Transfer of the construct may be performed by any of the methods mentioned which physically or chemically permeabilize the cell membrane.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an expression construct complexed with Lipofectamine (Gibco BRL).

Liposome-mediated nucleic acid delivery and expression of foreign DNA in vitro has been very successful (Nieolau and Sene, 1982; Fraley et al., 1979; Nicolau et al., 1987). Wong et al. (1980) demonstrated the feasibility of liposome-mediated delivery and expression of foreign DNA in cultured chick embryo, HeLa and hepatoma cells.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1.

Melloul et al. (1993) demonstrated transfection of both rat and human islet cells using liposomes made from the cationic lipid DOTAP, and Gainer et al. (1996) transfected mouse islets using Lipofectamine-DNA complexes.

In certain embodiments of the present invention, the expression construct is introduced into the cell via electroporation. Electroporation involves the exposure of a suspension of cells and DNA to a high-voltage electric discharge.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner. Examples of electroporation of islets include Soldevila et al. (1991) and PCT application WO 91/09939.

In other embodiments of the present invention, the expression construct is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

In another embodiment, the expression construct is delivered into the cell using DEAE-dextran followed by polyethylene glycol. In this manner, reporter plasmids were introduced into mouse myeloma and erythroleukemia cells (Gopal, 1985).

Another embodiment of the invention for transferring one or more naked DNA immortalizing or other expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Gainer et al. (1996) have transfected mouse islets with a luciferase gene/human immediate early promoter reporter construct, using biolistic particles accelerated by helium pressure.

Further embodiments of the present invention include the introduction of the expression construct by direct microinjection or sonication loading. Direct microinjection has been used to introduce nucleic acid constructs into Xenopus oocytes (Harland and Weintraub, 1985), and LTK⁻ fibroblasts have been transfected with the thymidine kinase gene by sonication loading (Fechheimer et al., 1987).

In certain embodiments of the present invention, the expression construct is introduced into the cell using adenovirus assisted transfection. Increased transfection efficiencies have been reported in cell systems using adenovirus coupled systems (Kelleher and Vos, 1994; Cotten et al., 1992; Curiel, 1994), and the inventors contemplate using the same technique to increase transfection efficiencies into human islets.

Still further constructs that may be employed to deliver the one or more immortalizing or other expression construct to the target cells are receptor-mediated delivery vehicles. These take advantage of the selective uptake of macromolecules by receptor-mediated endocytosis that will be occurring in the target cells. In view of the cell type-specific distribution of various receptors, this delivery method adds another degree of specificity to the present invention. Specific delivery in the context of another mammalian cell type is described by Wu and Wu (1993); incorporated herein by reference).

Certain receptor-mediated gene targeting vehicles comprise a cell receptor-specific ligand and a DNA-binding agent. Others comprise a cell receptor-specific ligand to which the DNA construct to be delivered has been operatively attached. Several ligands have been used for receptor-mediated gene transfer (Wu and Wu, 1987, 1988; Wagner et al., 1990; Ferkol et al., 1993; Perales et al., 1994; Myers, EPO 0273085), which establishes the operability of the technique. In the context of the present invention, the ligand will be chosen to correspond to a receptor specifically expressed on the neuroendocrine target cell population.

In other embodiments, the DNA delivery vehicle component of a cell-specific gene targeting vehicle may comprise a specific binding ligand in combination with a liposome. The nucleic acids to be delivered are housed within the liposome and the specific binding ligand is functionally incorporated into the liposome membrane. The liposome will thus specifically bind to the receptors of the target cell and deliver the contents to the cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the DNA delivery vehicle component of the targeted delivery vehicles may be a liposome itself, which will preferably comprise one or more lipids or glycoproteins that direct cell-specific binding. For example, Nicolau et al. (1987) employed lactosyl-ceramide, a galactose-terminal asialganglioside, incorporated into liposomes and observed an increase in the uptake of the insulin gene by hepatocytes. It is contemplated that the one or more immortalizing or other expression constructs of the present invention can be specifically delivered into the target cells in a similar manner.

2. Viral Infection

The vector expressing functional wild-type Pax4 to be used in an in vitro method of the invention or to be used in a pharmaceutical composition or a method of treatment in accordance with this invention, is particularly is a viral vector. Yet, also other systems for gene transfer as described above are envisaged in context of this invention. The viral vector may be a vector as discussed above but is particularly selected from the group consisting of an adenoviral vector, a retroviral vector or an lentiviral vector.

One of the preferred methods for delivery of expression constructs involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue-specific transforming construct that has been cloned therein.

The expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the E3, or both the E1 and E3 regions (Graham and Prevec, 1991). In nature, adenovirus can package approximately 105% of the wild-type genome (Ghosh-Choudhury et al., 1987), providing capacity for about 2 kb of extra DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of media. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of media, is added to the carrier (50 ml) in a 250 ml Erlenmeyer flask and left stationary, with occasional agitation, for 1 to 4 h. The media is then replaced with 50 ml of fresh media and shaking initiated. For virus production, cells are allowed to grow to about 80% confluence, after which time the media is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector preferred for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

Recombinant adenovirus and adeno-associated virus (see below) can both infect and transduce non-dividing human primary cells. Gene transfer efficiencies of approximately 70% for isolated rat islets have been demonstrated by the inventors (Becker et al., 1994a; Becker et al., 1994b; Becker et al., 1996) as well as by other investigators (Gainer et al., 1996).

Known adenoviral vector which are routinely employed in medical settings comprise Ad2 or Ad5. Herein below are adenoviral vectors described which may, inter alfa, be used in accordance with this invention. Such vectors are, inter alfa, adenoviral vector comprising a DNA as shown in SEQ ID NOS: 12 or 14. Accordingly, the invention also provides for an adenoviral vector system expressing function, wild-type Pax4, wherein said adenoviral vector system has the nucleic acid sequence as shown in SEQ ID NO: 15, which is a Ad2-vector expressing human wild-type Pax4 (pHP Ad2 Pax4; human) or as shown in SEQ ID NO. 16, which is a further adenoviral vector expressing human wild-type Pax4. Both adenoviral vectors also express a myc-tag. Additional “tags” are particularly useful in the vector systems provided herein since they allow for screening of cells and or tissues which have successfully been contacted with exogenous wild-type Pax4.

Adeno-associated virus (AAV) is an attractive vector system for use in the human cell transformation of the present invention as it has a high frequency of integration and it can infect nondividing cells, thus making it useful for delivery of genes into mammalian cells in tissue culture (Muzyczka, 1992). AAV has a broad host range for infectivity (Tratschin, et al., 1984; Laughlin, et al., 1986; Lebkowski, et al., 1988; McLaughlin, et al., 1988), which means it is applicable for use with human neuroendocrine cells, however, the tissue-specific promoter aspect of the present invention will ensure specific expression of the transforming construct in aspects of the invention where this is desired or required. Details concerning the generation and use of rAAV vectors are described in U.S. Pat. No. 5,139,941 and U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt, et al., 1994; Lebkowski, et al., 1988; Samulski, et al., 1989; Shelling and Smith, 1994; Yoder, et al., 1994; Zhou, et al., 1994; Hermonat and Muzyczka, 1984; Tratschin, et al., 1985; McLaughlin, et al., 1988) and genes involved in human diseases (Flotte, et al., 1992; Luo, et al., 1996; Ohi, et al., 1990; Walsh, et al., 1994; Wei, et al., 1994). Recently, an AAV vector has been approved for phase I human trials for the treatment of cystic fibrosis.

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski, et al., 1989; McLaughlin, et al., 1988; Kotin, et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994a; Clark et al., 1995). Cell lines carrying the rAAV DNA as an integrated provirus can also be used (Flotte et al., 1995).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975). Additional retroviral vectors contemplated for use in the present invention have been described (Osborne et al., 1990; Flowers et al., 1990; Stockschlaeder et al., 1991; Kiem et al., 1994; Bauer et al., 1995, Miller and Rosman, 1989; Miller, 1992; Miller et al., 1993; each incorporated herein by reference).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990). A preferred cell line is the PA317 cell line (Osborne et al., 1990).

A major determinant of virus titer is the number of packagable RNA transcripts per producer cell, which is dependent on the integrated proviral DNA copy number. Packaging cell lines are coated with viral envelope glycoproteins and are thus resistant to infection by virus of the same host range, but not virus of a different host range. This process is called interference. Therefore, recombinant retroviruses can shuttle back and forth between amphotropic and ecotropic packaging cell lines in a mixed culture (referred to as ping-ponging), thus leading to an increase in proviral DNA copy number and virus titer (Bestwick et al., 1988). Some drawbacks to the ping-pong process are that transfer of packaging functions between ecotropic and anphotropic lines can lead eventually to generation of replication-competent helper virus. Also, increasing numbers of cells express both ecotropic and amphotropic envelope proteins and are therefore resistant to further infection. Moreover, cells with large numbers of proviruses are unhealthy. Thus, there is an optimum period during the ping-pong process when virus titer is high and helper virus is absent. This time period is empirically determined and is relatively constant for a given ecotropic plus amphotropic packaging line combination.

Other viral vectors may be employed as expression constructs in the present invention. Vectors derived from viruses such as vaccinia virus (Ridgeway, 1988; Baichwal and Sugden, 1986; Coupar et al., 1988) and herpesviruses may be employed. They offer several attractive features for various mammalian cells (Friedmann, 1989; Ridgeway, 1988; Baichwal and Sugden, 1986; Coupar et al., 1988; Horwich et al., 1990). Lentivirus vectors are also contemplated for use in the present invention (Gallichan et al., 1998; Miyoshi et al., 1998; Kafri et al., 1999).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In still further embodiments of the present invention, the nucleic acids to be delivered are housed within an infective virus that has been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

### II. β-Cells and Cell Culturing

The invention also provides for a pancreatic β-cell obtained by the in vitro method described herein and documented in the appended examples. The pancreatic β-cells as obtained in accordance with the present invention are particularly useful in medical and pharmaceutical settings. Accordingly, the invention also provides for a pharmaceutical composition comprising a pancreatic β-cell as obtained by the in vitro method described herein or a pancreatic β-cell obtained by the method of the present invention. The β-cell obtained by the method of the present invention is, inter alia, characterized in that it is an adult β-cell which proliferates after contact with the herein described exogenous wild-type Pax4. Said β-cell may also comprise an expressed “tag”, like a myc-tag as provided, inter alia, by the adenoviral vectors described herein. These expressed “tags” are particularly useful in detection of β-cells which have successfully been contacted with exogenous wild-type Pax4 as described herein.

The (proliferating) pancreatic β-cell as obtained by the in vitro method of the invention is particularly used for the preparation of a pharmaceutical composition for transplantation and/or tissue replacement. Accordingly, the present invention also provides for the (medical/pharmaceutical) use of functional, wild-type Pax4 (as defined herein) for the preparation of a pharmaceutical composition for transplantation and/or tissue replacement. Said transplantation or tissue replacement is particularly to be carried out on a patient suffering from a pancreatic disease, most particularly from diabetes. The adult, proliferating β-cells as obtained by the method of the present invention may be employed in particular in transplantations and tissue gratings. It is of note that it is also envisaged to use these cells in devices, like encapsulation devices or immunobarrier devices (for example semi-permeable membrane devices). Accordingly, the β-cells as obtained by the method of the invention may also be comprised in therapy systems or devices to be implanted into, in subject in need of treatment, amelioration and/or prevention of a disorder/disease. These “micro-encapsulations” of the β-cells obtained by the method of the present invention is particularly useful in the treatment, amelioration and/or prevention of a pancreatic disorder, particularly of diabetes.

More generally, culture conditions may involve manipulating the following cell culture variables: media growth/survival factors (such as IGF-1, growth hormone, prolactin, PDGF, hepatocyte growth factor, and transferrin), media differentiation factors (such as TGF-β), media lipids, media metabolites (such as glucose, pyruvate, galactose, and amino acids), media serum (percentage serum, serum fraction, species of serum), gaseous exchange (ratio atmospheric O₂:CO₂, and media volume), physical form of the islets prior to plating (whole, dispersed, or cell sorted islet cells), and extracellular substrate for cellular attachment (such as laminin, collagen, matrigel, and HTB-9 bladder carcinoma derived matrix).

Media comprising one or more growth factors that stimulate the growth of the target cell and do not substantially stimulate growth of distinct cells in the cell population; i.e., act to induce preferential growth of the target cells rather than faster-growing, more hardy cells in the population, may be used to deplete fibroblasts. Examples include defined serum free conditions used for β-cells (Clark et al., 1990; incorporated herein by reference), or inclusion of growth or differentiation factors known to allow preferential growth of β-cells (WO95/29989; PCT/US99/00553; incorporated herein by reference). A commercially available medium, EGM Endothelial Cell Medium (Cambrex) is a preferred defined media.

Cells also may be induced to proliferate by initial infection with adenovirus or adeno-associated virus (AAV) comprising a gene that induces cellular proliferation, the gene being under the control of a promoter specific for the regulated secretory cell. The cells may alternatively be induced to proliferate by growth on a stimulatory cell matrix.

### III. Pharmaceutical Compositions

In context of the present invention, it is also envisaged that the herein described pharmaceutical compositions comprising the β-cells as obtained by the method of the invention be used in a method for treating a disorder characterized by insufficient pancreatic function in a subject. Methods of treatment comprise the introduction of the pharmaceutical composition described herein into a subject in need of such a treatment. Particularly, the subject is a human and the disorder to be treated is diabetes.

A. Pharmaceutically Acceptable Formulations

Where clinical applications are contemplated, it will be necessary to prepare pharmaceutical compositions of the cells in a form appropriate for transplant. The cells will generally be prepared as a composition that is essentially free of pyrogens, as well as other impurities that could be harmful to humans or animals.

One will generally desire to employ appropriate salts and buffers to render stable cells suitable for introduction into a patient. Aqueous compositions of the present invention comprise an effective amount of stable cells dispersed in a pharmaceutically acceptable carrier or aqueous medium, and preferably encapsulated.

The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and nil solvents, dispersion media, coatings, antibacterial and antifungal agents, isotonic and absorption delaying agents and the like. As used herein, this term is particularly intended to include biocompatible implantable devices and encapsulated cell populations. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

Under ordinary conditions of storage and use, the cell preparations may further contain a preservative to prevent growth of microorganisms. Intravenous vehicles include fluid and nutrient replenishers. Preservatives include antimicrobial agents, anti-oxidants, chelating agents and inert gases. The pH and exact concentration of the various components in the pharmaceutical are adjusted according to well-known parameters.

B. Cell-Based Delivery and Devices

The engineered cells of the present invention may be introduced into animals, including human subjects, with certain needs, such as patients with insulin-dependent diabetes. It should be pointed out that the studies of Madsen and coworkers have shown that implantation of poorly differentiated rat insulinoma cells into animals results in a return to a more differentiated state, marked by enhanced insulin secretion in response to metabolic fuels (Madsen et al, 1988). These studies suggest that exposure of engineered cell lines to the in vivo milieu may have some effects on their response(s) to secretagogues.

As discussed above, one method of administration involves the encapsulation of the engineered cells in a biocompatible coating. In this approach, the cells are entrapped in a capsular coating that protects the contents from immunological responses. One encapsulation technique involves encapsulation with alginate-polylysine-alginate. Capsules made employing this technique generally have a diameter of approximately 1 mm and should contain several hundred cells.

Cells may thus be implanted using the alginate-polylysine encapsulation technique of O′Shea and Sun (1986), with modifications, as later described by Fritschy et al. (1991; both references incorporated herein by reference). The engineered cells are suspended in 1.3% sodium alginate and encapsulated by extrusion of drops of the cell/alginate suspension through a syringe into CaCl₂. After several washing steps, the droplets are suspended in polylysine and re-washed. The alginate within the capsules is then reliquified by suspension in 1 mM EGTA and then rewashed with Krebs balanced salt buffer.

An alternative approach is to seed Amicon fibers with stable cells of the present invention. The cells become enmeshed in the fibers, which are semipermeable, and are thus protected in a manner similar to the micro encapsulates (Altman et al., 1986; incorporated herein by reference). After successful encapsulation or fiber seeding, the cells may be implanted intraperitoneally, usually by injection into the peritoneal cavity through a large gauge needle (23 gauge).

A variety of other encapsulation technologies have been developed that are applicable to the practice of the present invention (see, e.g., Lacy et al., 1991; Sullivan et al., 1991; WO 91/10470; WO 91/10425; WO 90/15637; WO 90/02580; U.S. Pat. No. 5,011,472; U.S. Pat. No. 4,892,538; and WO 89/01967; each of the foregoing being incorporated by reference).

Lacy et. al. (1991) encapsulated rat islets in hollow acrylic fibers and immobilized these in alginate hydrogel. Following intraperitoneal transplantation of the encapsulated islets into diabetic mice, normoglycemia was reportedly restored. Similar results were also obtained using subcutaneous implants that had an appropriately constructed outer surface on the fibers. It is therefore contemplated that engineered cells of the present invention may also be straightforwardly “transplanted” into a mammal by similar subcutaneous injection.

Sullivan et. al. (1991) reported the development of a biohybrid perfused “artificial pancreas,” which encapsulates islet tissue in a selectively permeable membrane. In these studies, a tubular semi-permeable membrane was coiled inside a protective housing to provide a compartment for the islet cells. Each end of the membrane was then connected to an arterial polytetrafluoroethylene (PTFE) graft that extended beyond the housing and joined the device to the vascular system as an arteriovenous shunt. The implantation of such a device containing islet allografts into pancreatectomized dogs was reported to result in the control of fasting glucose levels in 6/10 animals. Grafts of this type encapsulating engineered cells could also be used in accordance with the present invention.

The company Cytotherapeutics has developed encapsulation technologies that are now commercially available that are envisioned for use in the application of the present invention. A vascular device has also been developed by Biohybrid, of Shrewsbury, Mass., that can be used with the technology of the present invention. Other implantable containment apparati contemplated for use with in the application of the present invention are described in U.S. Pat. Nos. 5,626,561, 5,787,900 and 5,843,069, each of which are incorporated herein by reference.

Implantation employing such encapsulation techniques provides various advantages. For example, transplantation of islets into animal models of diabetes by this method has been shown to significantly increase the period of normal glycemic control, by prolonging xenograft survival compared to unencapsulated islets (O'Shea and Sun, 1986; Fritschy et 1991). Also, encapsulation will prevent uncontrolled proliferation of clonal cells. Capsules containing cells are implanted (approximately 1,000-10,000/animal) intraperitoneally and blood samples taken daily for monitoring of blood glucose and insulin.

An alternate approach to encapsulation is to simply inject glucose-sensing cells into the scapular region or peritoneal cavity (Sato et al., 1962). Implantation by this approach may circumvent problems with viability or function, at least for the short term, that may be encountered with the encapsulation strategy.

### IV. Treatments

The term “treatment” in accordance with this invention also comprises the amelioration or even prevention of a disorder/disease, e.g., a pancreatic disorder, in particular diabetes. The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The present invention is directed towards treating patients with medical conditions relating to a disorder of the pancreas. Accordingly, a treatment of the invention would involve preventing, inhibiting or relieving any medical condition related to pancreatic disorders, particularly diabetes. The “treatment” of the present invention is in particular achieved by tissue engineering (of pancreatic cells or islets) and transplantation approaches. Said transplantations comprise, but are not limited to, cell and tissue grafting, islet transplantations and tissue replacement therapies. As discussed herein, the transplantation is not limited to human-human transplantations (homo-transplantations) but also comprises animal-human transplantations (xenotransplantation). Particularly are, however, human-human transplantations and most particularly, the method of the invention is employed to in vitro proliferate adult β-cells obtained from the patient to be treated and to re-implant the proliferated β-cells obtained by the inventive method. Most particularly, the transplantation is carried out in accordance with the Edmonton protocol. In accordance with this invention it is envisaged that wild-type Pax4 encoding nucleic acid molecules or expressed wild-type Pax4 (e.g., mRNA or protein) be employed to stimulate proliferation of pancreatic cells, e.g., β-cells ex vivo.

An effective amount of the stable cells is determined based on the intended goal. The term “unit dose” refers to a physically discrete unit suitable for use in a subject, each unit containing a predetermined quantity of the cell composition calculated to produce the desired response in association with its administration, i.e., the appropriate route and treatment regimen. The quantity to be administered, both according to number of treatments and unit dose, depends on the subject to be treated, the state of the subject, and the protection desired. Precise amounts of the therapeutic composition also depend on the judgment of the practitioner and are peculiar to each individual.

### V. Adjunct Therapies and Procedures

In accordance with the present invention, it may prove advantageous to combine the methods disclosed herein with adjunct therapies or procedures to enhance the overall therapeutic effect. Such therapies and procedures are set forth in general, below. A skilled physician will be apprised of the most appropriate fashion in which these therapies and procedures may be employed.

A. Supplemental Insulin Therapy

The present invention, though designed to eliminate the need for other therapies, may work well in combination with traditional insulin supplementation. Such therapies should be tailored specifically for the individual patient given their current clinical situation, and particularly in light of the extent to which transplanted cells can provide insulin. The following are general guidelines for typical a “monotherapy” using insulin supplementation by injection.

Insulin can be injected in the thighs, abdomen, upper arms or gluteal region. In children, the thighs or the abdomen are preferred. These offer a large area for frequent site rotation and are easily accessible for self-injection. Insulin injected in the abdomen is absorbed rapidly while from the thigh it is absorbed more slowly. Hence, patients should not switch from one area to the other at random. The abdomen should be used for the time of the day when a short interval between injection and meal is desired (usually pre-breakfast when the child may be in a hurry to go to school) and the thigh when the patient can wait 30 minutes after injection for his meal (usually pre-dinner). Within the selected area systematic site rotation must be practiced so that not more than one or two injections a month are given at any single spot. If site rotation is not practiced, fatty lumps known as lipohypertrophy may develop at frequently injected sites. These lumps are cosmetically unacceptable and, what is more important, insulin absorption from these regions is highly erratic.

Before injecting insulin, the selected site should be cleaned with alcohol. Injecting before the spirit evaporates can prove to be quite painful. The syringe is held like a pen in one hand, pinching up the skin between the thumb and index finger of the other hand, and inserting the needle through the skin at an angle of 45-90° to the surface. The piston is pushed down to inject insulin into the subcutaneous space (the space between the skin and muscle), then one waits for a few seconds after which release the pinched up skin before withdrawing the needle. The injection site should not be massaged.

For day-to-day management of diabetes, a combination of short acting and intermediate acting insulin is used. Some children in the first year after onset of diabetes may remain well controlled on a single injection of insulin each day. However, most diabetic children will require 2, 3 or even 4 shots of insulin a day for good control. A doctor should decide which regimen is best suited.

One injection regimen: A single injection comprising a mix of short acting and intermediate acting insulin (mixed in the same syringe) in 1:3 or 1:4 proportion is taken 20 to 30 minutes before breakfast. The usual total starting dose is 0.5 to 1.0 units/kg body weight per day. This regimen has three disadvantages: (1) all meals must be consumed at fixed times; (2) since the entire quantity of insulin is given at one time, a single large peak of insulin action is seen during the late and early evening hours making one prone to hyopglycemia at this time; (3) as the action of intermediate acting insulin rarely lasts beyond 16-18 hours, the patient's body remains underinsulinized during the early morning hours, the period during which insulin requirement in the body is actually the highest.

Two-injection regimen: This regimen is fairly popular. Two shots of insulin are taken one before breakfast (⅔ of the total dose) and the other before dinner (⅓ of the total dose). Each is a combination of short acting and intermediate acting insulin in the ratio of 1:2 or 1:3 for the morning dose, and 1:2 or 1:1 for the evening dose. With this regimen the disadvantages of the single injection regimen are partly rectified. Some flexibility is possible for the evening meal. Further, as the total days' insulin is split, single large peaks of insulin action do not occur hence risk of hypoglycemia is reduced and one remains more or less evenly insulinized throughout the day. On this regimen, if the pre-breakfast blood glucose is high, while the 3 a.m. level is low, then the evening dose may need to be split so as to provide short acting insulin before dinner and intermediate acting insulin at bedtime.

Multi-dose insulin regimens: The body normally produces insulin in a basal-bolus manner, i.e., there is a constant basal secretion unrelated to meal intake and superimposed on this there is bolus insulin release in response to each meal. Multi-dose insulin regimens were devised to mimic this physiological pattern of insulin production. Short acting insulin is taken before each major meal (breakfast, lunch and dinner) to provide “bolus insulin” and intermediate acting insulin is administered once or twice a day for “basal insulin.” Usually bolus insulin comprises 60% of the total dose and basal insulin makes up the remaining 40%. With this regimen you have a lot of flexibility. Both the timing as well as the quantity of each meal can be altered as desired by making appropriate alterations in the bolus insulin doses. To take maximum advantage of this regimen, one should learn “carbohydrate counting” and work out carbohydrate:insulin ratio—the number of grams of carbohydrate for which the body needs 1 unit of insulin.

B. Immunosuppressive Therapy

In accordance with the present invention, it may prove necessary to deliver an immunosuppressive therapy to a transplant recipient to prevent graft rejection. A general approach to transplant immunosuppression is to combine agents in small doses so as to get an added immunosuppressive effect, but without individual side effects of the different drugs. Commonly used agents include azathioprine, corticosteroids and cyclosporin are combined in a variety of protocols. Each has different side effects: corticosteroids stunt growth and cause a round face and impair the healing of wounds; azathioprine can inhibit the bone marrow and cause anaemia and a low white cell count; cyclosporin can cause increase in growth of hair and damage the kidney. However, when these three agents are used together in reduced doses, the patient can generally tolerate the immunosuppression quite well.

Unfortunately, acute rejection crises can still occur and are usually treated with a short course of high dose steroids or anti-lymphocyte globulin preparations. This powerful immunosuppression for rejection may lead to infection particularly with viruses, causing severe cold sores and may activate cytomegalic virus infection which can cause a temperature and specific bad effects on a variety of different organs. All immunosuppression predisposes a patient to infection and tumour formation.

Despite these difficulties, most patients tolerate organ grafts and, after a time, can be maintained with relatively low dosage of immunosuppression. With few exceptions however, stopping immunosuppression usually leads to acute rejection and chronic rejection which is difficult to detect and can occur insidiously after years of good function of a graft. Thus, the search for new immunosuppressives such as FK506, celcept mycophenolate mofital and rapamycin is important.

C. Pro-Angiogenic Therapy

Pro-angiogenic therapy, also known as “therapeutic angiogenesis,” uses angiogenic growth factors or gene therapy to stimulate blood vessel growth in tissues that require an improved blood supply. While angiogenesis is normally activated by hypoxia (decreased oxygen), many afflicted tissues are unable to respond adequately to reverse the disease processes and prevent tissue damage.

Currently, a variety of angiogenesis-stimulating modalities are being tested in clinical trials sponsored by biotechnology and pharmaceutical companies, medical centers, and the National Institutes of Health. The most prevalently discussed pro-angiogenic therapy is the use of VEGF. Another involves a topical gel of recombinant platelet-derived growth factor (rhPDGF-BB).

D. Monitoring Glucose Levels

Any person suffering from diabetes will be very familiar with the need to regularly measure blood glucose levels. Blood glucose level is the amount of glucose, or sugar, in the blood. It is also is referred to as “serum glucose level.” Normally, blood glucose levels stay within fairly narrow limits throughout the day (4 to 8 mmol/l), but are often higher after meals and usually lowest in the morning. Unfortunately, when a person has diabetes, their blood glucose level sometimes moves outside these limits. Thus, much of a diabetic's challenge is to When one suffers from diabetes, it is important that glucose level be as near normal as possible. Stable blood glucose significantly reduces the risk of developing late-stage diabetic complications, which start to appear 10 to 15 years after diagnosis with Type 1 diabetes, and often less than 10 years after diagnosis with Type 2 diabetes.

Blood glucose levels can be measured very simply and quickly with a home blood glucose level testing kit, consisting of a measuring device itself and a test strip. To check blood glucose level, a small amount of blood is placed on the test strip, which is then placed into the device. After about 30 seconds, the device displays the blood glucose level. The best way to take a blood sample is by pricking the finger with a lancet. Ideal values are (a) 4 to 7 mmol/l before meals, (b) less than 10 mmol/l one-and-a-half hours after meals; and (c) around 8 mmol/1 at bedtime.

People who have Type 1 diabetes should measure their blood glucose level once a day, either in the morning before breakfast or at bedtime. In addition, a 24-hour profile should be performed a couple of times a week (measuring blood glucose levels before each meal and before bed). People who have Type 2 diabetes and are being treated with insulin should also follow the schedule above. People who have Type 2 diabetes and who are being treated with tablets or a special diet should measure their blood glucose levels once or twice a week, either before meals or one-and-a-half hours after a meal. They should also perform a 24-hour profile once or twice a month.

The main advantage for measuring blood glucose levels of insulin-treated diabetics in the morning is that adjusted amounts of insulin can be taken if the blood glucose level is high or low, thereby reducing the risk of developing late-stage diabetic complications. Similarly, the blood glucose level at bedtime should be between 7 and 10 mmol/l. If blood glucose is very low or very high at bedtime, there may be a need to adjust food intake or insulin dose. Blood glucose should also be measured any time the patient does not feel well, or think blood glucose is either too high or too low. People who have Type 1 diabetes with a high level of glucose in their blood (more than 20 mmol/l), in addition to sugar traces in the urine, should check for ketone bodies in their urine, using a urine strip. If ketone bodies are present, it is a warning signal that they either have, or may develop, diabetic acidosis.

### VI. Kits

The invention also relates to a kit comprising a vector system as discussed above or a gene delivery device, wherein said vector system or gene delivery device leads to the expression of functional, wild-type Pax4. Particularly, said kit comprises an adenoviral vector as defined above. The inventive kit may include buffers and substrates for reporter genes that may be present in recombinant Pax4 molecules or vectors of the invention. The kit of the invention may advantageously be used for carrying out any one of the methods of the invention and could be, inter alia, employed in a variety of applications referred to herein, e.g., in the medical field, the pharmaceutical field or as research tool. The parts of the kit of the invention can be packaged individually in vials or in combination in containers or multicontainer units. Manufacture of the kit follows particularly standard procedures which are known to the person skilled in the art.

### VII. Examples

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Material and Methods Used in this Study

Rat islet isolation and culture. 7-week-old male Wistar rats (250 g) were purchased from Elevage Janvier (Le Genest-St-Isle, France). Pancreatic islets were isolated by collagenase digestion, handpicked and maintained in 11.5 mM glucose/RPMI-1640 (Invitrogen, Basel, Switzerland) supplemented with 10% fetal calf serum (FCS; Brunschwig AG, Basel, Switzerland), 100 Units/ml penicillin, 100 mg/ml streptomycin and 100 μg/ml gentamycin (Sigma Basel, Switzerland). In several instances, islets were exposed to 0.1, 0.5 and 2 nM of activin A, betacellulin and TGFβ1 (Sigma, Basel, Switzerland) for 24 h.

Human isolation and culture. Freshly isolated human islets, obtained from the Cell Isolation and Transplantation Laboratory in Geneva, were maintained in CMRL-1066 supplemented with 10% FCS, 100 Units/ml penicillin, 100 μg/ml streptomycin and 100 μg/ml gentamycin (Fournier, 1996; Janjic, 1996). Islets were treated with 11 mM or 25 mM glucose, 0.5 nM betacellulin, activin A or TGF-β1 for 24 and 48 hours. Total RNA was isolated using RNeasy mini kit as described by the manufacturer (Quiagen). Pax4 and cyclophilin mRNA levels were assessed by quantitative RT-PCR using an ABI 7000 Sequence Detection System (Applera Europe). Alternatively, human islets were transduced with either AdCaLacZ or AdCMVPax4IRESGFP and proliferation was assessed by BrdU incorporation.

Plasmid construction. The full-length mouse Pax4 cDNA was amplified by polymerase chain reaction (PCR) and the PCR product was subcloned into an expression vector pGEM-T Easy (Promega). An EcoRI fragment containing Pax4 was then transferred into the expression vector pcDNA3.1/myc-His (Invitrogen). The inventors used the myc-tagged fusion protein for immunofluorescent detection of Pax4, because anti-Pax4-specific antibodies failed to detect the transcription factor. The Pax4-myc wild-type (wt) was subjected to mutagenesis using the oligonucleotides 5′-CGAGTACTTTGGGCACTTC-3′ (SEQ ID NO:17) and 5′-GAAGTGCCCAAAGTACTCG-3′ (SEQ ID NO:18), the Turbo Pfu polymerase (Stratagene) and the restriction enzyme DpnI to generate mutant Pax4-myc R129W (arginine at codon 129 to tryptophan). The mouse mutant R129W of Pax4 gene was described to correspond to the human mutation R121W (Shimajiri, 2001).

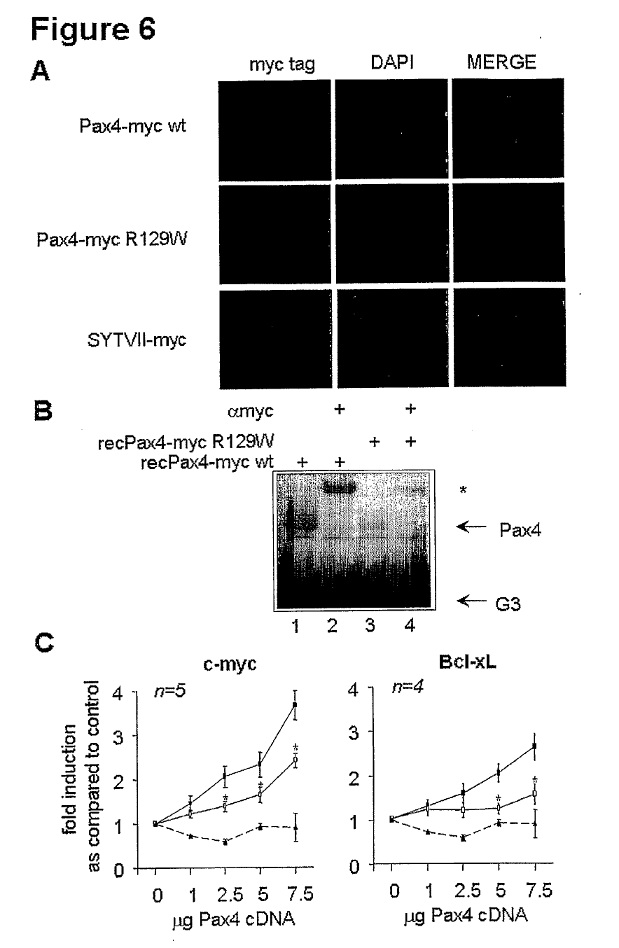

Adenoviral infection of rat islets. The recombinant adenoviruses AdCMVPax4IRESGFP, AdCMVNgn3IRESGFP and AdCMVPax6 were generated using standard methods; see also FIG. 6. AdCAlacZ, containing the bacterial β-galactosidase cDNA, was used as control (Ishihara, 1999). Twelve hours post-isolation, islets were infected with various amounts of recombinant adenoviruses (as indicated in FIGS.) for 90 min, washed and cultured in RPMI 1640 medium supplemented with 10% FCS. The overexpression of Pax4 in infected islets was monitored daily by EGFP expression visualized by fluorescent microscopy.

Generation of further adenoviral vectors comprising wildtype and mutant Pax4. The mouse Pax4 cDNA was amplified by PCR and cloned into the pcDNA-3 vector (Invitrogen) in frame with the c-myc/6-Histidine (6-HIS) tag sequence. This carboxy-terminal end tag allowed for the detection of the recombinant protein by immunofluorescence using an antibody against the c-myc epitope. Site directed mutagenesis was performed on this construct to generate the R129W mutant. This mutation located in the paired DNA binding domain corresponds to the human R121W mutation which has been associated with type 2 diabetes. The wild-type and mutant Pax4/c-myc/6-HIS cDNAs were then subcloned into the pTRE-Shuttle2 vector harbouring a tetracycline-inducible cytomegalovirus (CMV) promoter (Clontech). The inducible cassettes were subsequently transferred into the Adeno-X viral DNA to generate recombinant adenoviruses (Ad-mPax4-myc WT and Ad-mPax4-myc R129W). Further adenoviral vectors comprise the pHVAd2-Pax4 vector, generated as follows: Pax4 cDNA has been isolated by standard molecular biology cloning techniques and cloned into Bam HI and Hind III restriction sites of the first generation adenoviral vector pACCMV.pLpA (Becker, 1994) which contains the CMV promoter and the SV40 poly-adenylation (polA) signal. In a second step the CMV-Pax4-SV40 polA transgene has been isolated by Not I restriction digest, blunted and cloned into the Eco RV restriction site of pHVAd2 (provided by DeveloGen Berlin).

The herein provided Ad-Pax4-myc construct was generated as follows. The Myc-His tag added at the end of mouse Pax4 nucleic acid sequence plus linker sequences, leading to the following construct:

ATG . . . ctcaaactgg cca)
pax4-
(atc act agt gaa ttc tgc aga tat cca gca cag tgg
cgg ccg ctc gag tct aga ggg ccc ttc)
Linker-

(SEQ ID NO: 19)

(GAA CAA AAA CTC ATC TCA GAA GAG GAT CTG)
myc epitope-

(SEQ ID NO: 20)

(AAT ATG CAT ACC GGT CAT CAT CAC CAT CAC CAT)
PolyHIS tag

This cassette was cloned into position 787 of pTRE-Shuttle2 (EcoRV site). This construct was digested with enzymes Cad (position 18 of vector) and SceI (position 1608 of vector) and subcloned into CeuI and SceI predigested pAdeno-X viral DNA (position 21 and 57 respectively)

Infection studies on isolated human islets. Freshly isolated human islets, obtained from the Cell Isolation and Transplantation Laboratory in Geneva, were maintained for 48 hours in CMRL-1066 supplemented with 10% FCS, 100 Units/ml penicillin, 100 μg/ml streptomycin and 100 μg/ml gentamycin. Partially trypsinized islets were then infected with either Ad-mPax4-myc WT or Ad-mPax4-myc R129W along with the adenoviral construct harbouring the tetracycline transcriptional activator (Ad-X Tet-On). Cells were rinsed 90 minutes post infection and replenished with fresh media supplemented with or without doxycycline (1 μg/ml) and BrdU (10 μM). Doxycycline-dependent activation of PAX4 as well as cell proliferation was then assessed 48 hours later by immunohistochemistry using antibodies raised against the c-myc epitope and BrdU, respectively.

Quantitative real-time RT-PCR. Total RNA from 50 islets was extracted using the Trizol reagent (Invitrogen) and 2 μg were converted into cDNA as previously described (Gauthier et al., 1999b). Primers for cyclophilin, somatostatin, glucagon, insulin, Pdx1, c-myc, Id2, Bcl-xL, Bcl-2, Pax4 and caspase 3 were designed using the Primer Express Software (Applera Europe, Rotkreuz, Switzerland) and sequences can be obtained upon request. Quantitative real-time PCR was performed using an ABI 7000 Sequence Detection System (Applera Europe) and PCR products were quantified fluorometrically using the SYBR Green Core Reagent kit. Three distinct amplifications derived from 4 independent experiments were performed in duplicate for each transcript and mean values were normalized to the mean value of the reference mRNA cyclophilin. An RNA control sample was used with each real time PCR experiment containing cyclophilin primers to control for genomic DNA contamination. Quantification was achieved by the standard curve method as described by Applera Europe.

Transient transfection assays. The c-myc (pDEL-1-Luc), Bcl-xL (Bcl-xL/515) and telomerase (pTERT-luc) gene promoter luciferase reporter constructs were kindly provided by Dr B. Vogelstein (The Johns Hopkins Oncology Center, Baltimore, Md.), Dr B Schaefer (University of Zurich, Zurich, CH) and Dr R Dalla-Favera (Columbia University, New York, N.Y.), respectively. The BHK-21 cell line was transiently transfected using the calcium phosphate precipitation technique as described previously (Gauthier, 1999a). The pSV-β-Galactosidase control vector (Promega) was used as internal control to normalize for transfection efficiency. Values presented for each set of experiments correspond to the mean and standard deviation of at least three individual transfections performed in duplicates. Results are presented as fold induction of the control sample obtained from cells transfected with empty expression vector.

Nuclear extract preparation and electrophoretic mobility shift assay (EMSA). Nuclear protein extracts were prepared from AdCaLacZ or AdCMVPax4IRESGFP-infected islets according to the protocol of Schreiber et al. (Schreiber, 1988). DNA binding assays using a radiolabeled glucagon promoter G3 element were performed as described previously (Gauthier, 2002). Recombinant Pax4 as well as Pax6 were prepared using an in vitro transcription and translation system as described by the manufacturer (Promega Inc.). Antibodies generated against Pax4 and Pax6 were kindly provided by Dr M. S. German (UCSF, San Francisco Calif., USA) and Dr S. Saule (Institut Curie, Orsay Cedex, France), respectively.

Hormone radioimmunoassays. Islets were washed in Krebs-Ringer-bicarbonate-HEPES buffer (KRBH) and incubated at 37° C. for 60 min in the same buffer supplemented with 2.5 mM glucose. Subsequently, insulin secretion from 15 matched-size islets/condition was measured over a period of 30 min in KRBH containing the indicated stimulators. Total hormone content was extracted using acid ethanol. Total and secreted insulin was detected by radioimmunoassay as described previously (Maechler and Wollheim, 1999). Secreted insulin was expressed as a percentage of total cellular insulin content. For glucagon radioimmunoassays, a protocol was adapted from that of Salehi et al. (Salehi, 1999) as follows: 50 μl samples of diluted (1:400) acid ethanol extracts were kept for 24 h at 4° C. in the presence of an equal volume of anti-glucagon (Dako Diagnostics AG, Zug, Switzerland; diluted 1:150 in PBS-EDTA, containing 0.5% BSA). Glucagon I¹²⁵ tracer (50 μl) was then added and assays were maintained for a further 48 h at 4° C. Subsequently, PBS-EDTA containing 4% BSA (100 μl) was included and the reaction was kept at 4° C. for an additional 45 min. Free Glucagon I¹²⁵ was then separated from bound tracer through precipitation with charcoal as described for insulin (Maechler and Wollheim, 1999). A standard curve was generated in parallel using purified rat glucagon 1-29 (Bachem AG, Switzerland) diluted in the range of 25-400 pg.

Glucose oxidation. Carbon dioxide production derived from glucose oxidation was measured using the multiwell ¹⁴CO₂-capture assay developed by Collins and colleagues (Collins, 1998). Radioactive carbon dioxide trapped in filter paper was measured using a Beckman LS6500 Scintillation counter (Beckman Coulter International, Nyon, Switzerland) and results were expressed as nmole of glucose/mg protein/hour.

ATP measurements. Total cellular ATP was measured after 10 min incubation in 2.5 mM glucose using a bioluminescence assay kit according to the manufacturer's recommendations (HS II, Roche Diagnostic, Rotkreuz, Switzerland). Alterations in cytosolic ATP levels in response to 16.5 mM glucose were monitored using the adenoviral construct AdCAGLuc encoding the ATP-sensitive bioluminescence probe luciferase (Ishihara, 2003). Changes in ATP were then recorded using a FLUOstar Optima apparatus (BMG Labtechnologies) as previously described by Merglen et al. (Merglen, 2004).

Mitochondrial calcium measurements. Islets were infected with rAdRIPmAQ (4.8×10⁸ pfu/ml) and either AdCaLacZ or AdCMVPax4IRESGFP (2.4×10⁷ pfu/ml) for 90 min. Islets were then washed and immediately seeded onto A431 extracellular matrix-coated 15-mm diameter Thermanox™ coverslips (Nunc), at a density of approx 80 islets per coverslip, and cultured in complete medium for 65 hr to maximise adherence and allow for the expression of recombinant protein (Ishihara, 2003). The mtAq was reconstituted with coelenterazine (5 μM, Molecular Probes) in glucose/serum-free RPMI medium for 2-4 h immediately prior to the experiment. Cover slips were then placed in a sealed, thermostatted (37° C.) chamber, 5 mm from a photonmultiplier, which was used to detect emitted luminescence, as previously described (Kennedy, 1996). Islets were superfused (1.0 ml min⁻¹) with KRBH supplemented with either glucose (16.7 mM) or KCl (60 mM) where indicated. Luminesence output was recorded every second using a photon-counting board (EMI C660) after a 30 min equilibration period to establish the baseline. Recorded counts were converted to [Ca²⁺]ₘ as published elsewhere (Challet, 2001).

Immunohistochemistry and cell proliferation assay. Isolated rat islets were infected with recombinant adenoviruses and subsequently dissociated by trypsin-EDTA treatment. Single cells were cultured on polyornithine-treated glass cover slips for two days, washed with PBS and fixed in 4% paraformaldehyde in PBS for 20 min at room temperature. Immunochemical detection of β-cells was performed as previously described (Ishihara, 2003) using a polyclonal guinea pig anti-insulin antibody (dilution 1:1000; Sigma). The antibody was visualized using a Texas Red dye-conjugated anti-guinea pig IgG (Milan analytica AG, La Roche, Switzerland; dilution 1:400). Nuclei were then stained with 4′,6-diamidino-2-phenylindole (DAPI, 10 μg/ml; Sigma) for 3 min. Cover slips were rinsed with PBS and mounted using DAKO fluorescent mounting medium. Images were obtained using a Zeiss Axiophot 1 equipped with an Axiocam color CCD camera.

Cellular proliferation was estimated using an immunohistochemical assay kit as described by the manufacturer (Roche Diagnostics, Rotkreuz, Switzerland). Seven h prior to fixation with ethanol, infected or mitogen treated-cells were labeled with 10 μM BrdU. Cells were co-stained for insulin, as described above, and for BrdU using a monoclonal anti-BrdU mouse IgG (Sigma). BrdU positive cells were visualized after incubation with fluorescein isothiocyanate (FITC)-conjugated anti mouse IgG. β-cells immunostained for both insulin and BrdU were counted using a Zeiss microscope under high magnification (×400). Results are expressed as a percentage of BrdU/insulin positive cells over the total amount of insulin positive cells for each condition. Recombinant Pax4 WT or R129W myc tagged proteins were visualized using an anti-myc antibody (Invitrogen).

Statistical analysis. Results are expressed as mean+/−SE. Where indicated, the statistical significance of the differences between groups was estimated by Student's impaired t-test. * and ** indicate statistical significance with p<0.05 and p<0.01, respectively.

### Example 2

### Activin A and Betacellulin Increase Pax4 Gene Transcription as Well as β-Cell Proliferation in Rat Islets

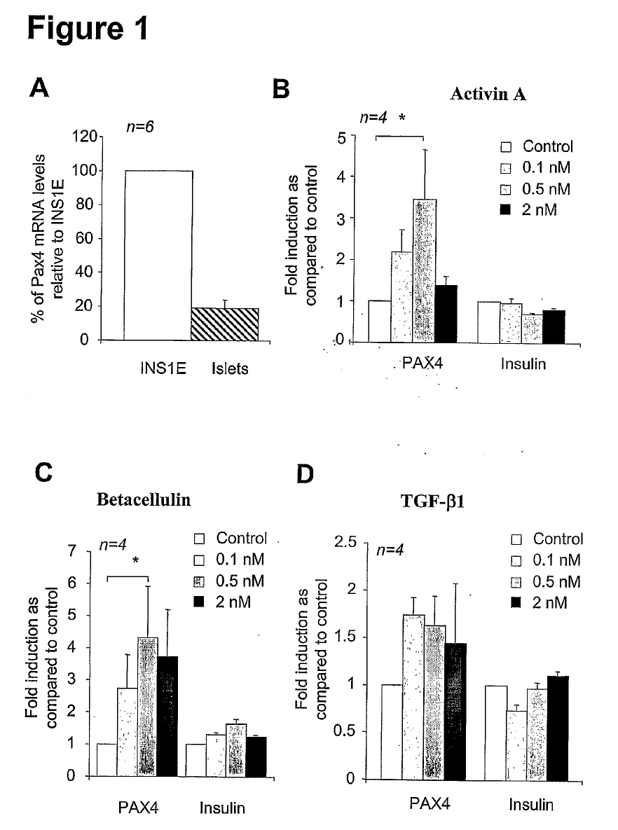

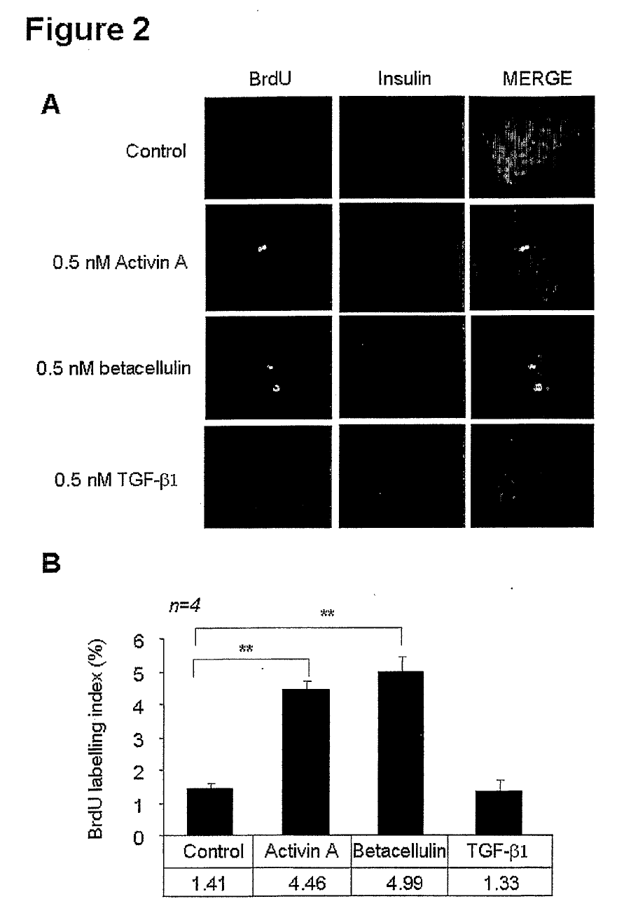

As an initial step towards understanding factors that may influence Pax4 expression and thereby impact endocrine cell mass, the inventors investigated the response of the pax4 gene to mitogens such as activin A and betacellulin in rat islets (Demeterco, 2000). The inventors first established the relative basal mRNA expression level of the transcription factor in rat islets and INS-1E cells. Similar to other insulinomas, the INS-1E cell line expresses Pax4 and therefore provides a suitable reference to measure relative levels of the transcript (Miyamoto, 2001). Pax4 mRNA was detected in rat islets at levels 80% lower than those found in INS-1E cells thus confirming expression of the transcription factor in adult tissue (FIG. 1A). Treatment of islets for 24 h with a range of concentrations of either activin A or betacellulin resulted in a dose-dependent increase of Pax4 mRNA levels. Maximal induction was observed with 0.5 nM of activin A or betacellulin that elicited a 3.5- and 4-fold increase in Pax4 mRNA, respectively (FIGS. 1B and C). In contrast, the related factor TGF-β1 had no significant effect on Pax4 in islets (Ueda, 2000) (FIG. 1D). Of note, insulin mRNA levels were unaffected by both treatments (FIGS. 1B-D) (Ueda, 2000). In parallel, activin A and betacellulin-induced proliferation was measured by BrdU incorporation. This approach was selected because of its ability to discriminate between β-cells proliferation and that of other cell type, in contrast to the [³H] thymidine incorporation method. FIG. 2A illustrates a representative experiment in which insulin and BrdU were immuno-detected and co-stained in dispersed islet cells. Since Pax4 is predominantly localized to β-cells, only insulin-positive cells were included in the BrdU labelling index calculation. Both growth factors were found to increase β-cell proliferation by approximately 2-fold (control, 1.41±0.2; activin A, 4.46±0.3; betacellulin, 4.99±0.4) while TGF-β1-treated islets remained quiescent (1.33±0.3) (FIG. 2B). Taken together, these results suggest that stimulation of Pax4 gene expression by activin A and betacellulin correlates with islet proliferation induced by the two mitogens.

### Example 3

### Adenovirus-Mediated Pax4 Overexpression in Rat Islets Induces β-Cell Proliferation

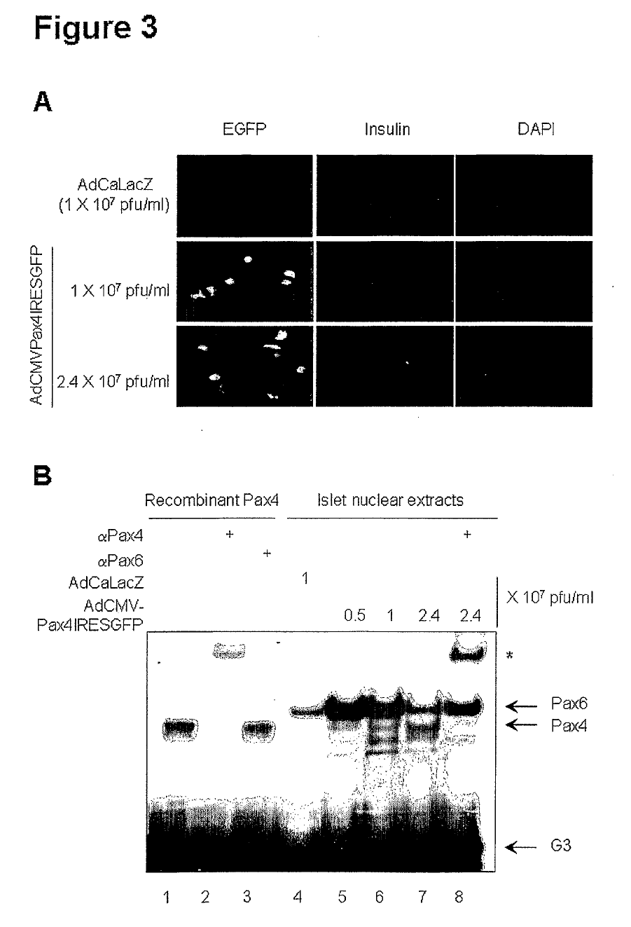

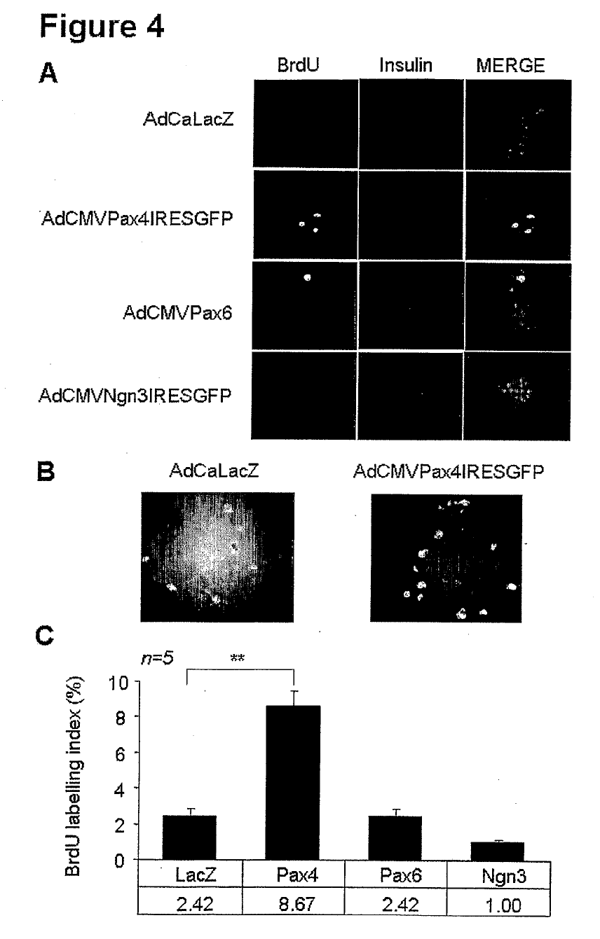

To evaluate the importance of Pax4 in β-cell replication, isolated rat pancreatic islets were infected with a CMV promoter driven Pax4/GFP-expressing adenovirus (AdCMVPax4IRESGFP) or control adenovirus (AdCAlacZ). Since the antibody against Pax4 is unable to detect the protein by immunohistochemistry or by Western blotting (data not shown), the inventors monitored its overexpression via the reporter green fluorescent protein (GFP) co-translated from a bi-cistronic transcript containing an internal ribosomal entry sequence (IRES). Immunohistochemical studies revealed that approximately 25% and 50% of β-cells expressed GFP 48 h after infection with 1 and 2.4×10⁷ pfu/ml of AdCMVPax4IRESGFP, respectively (FIG. 3A). Nuclear fragmentation was absent in transduced cells indicating that viral infection did not affect viability or apoptosis. Consistent with GFP expression, Pax4 transcript was estimated to reach levels 22±5-fold higher (n=3) than those found in control AdCALacZ-infected islets (data not shown). In order to confirm the production of a functional protein, the inventors performed electromobility shift assays (EMSA) using a radiolabeled G3 element of the glucagon gene promoter, which has previously been shown to interact with both Pax4 and Pax6 (Ritz-Laser et al., 2002). A single predominant complex corresponding to Pax6 was detected in nuclear extracts derived from AdCaLacZ-infected islets (FIG. 3B, lane 4). A second complex of similar migration pattern to that produced by recombinant Pax4 was generated in islet infected with increasing amounts of AdCMVPax4IRESGFP (FIG. 3B lanes 1, 5, 6 and 7). The addition of Pax4 antiserum specifically abolished this complex confirming the binding of Pax4 to this site (FIG. 3B lane 2 and 8). The inventors next evaluated the capacity of Pax4 to promote islet proliferation by measuring BrdU incorporation in either dispersed cells (FIG. 4A) or whole islets (FIG. 4B). Quantification revealed a 3.5-fold increase in BrdU labelling of β-cells expressing Pax4 as compared to AdCALacZ-transduced islets (8.67±0.8% versus 2.42±0.5%, FIG. 4C). To confirm the specificity of Pax4-associated β-cell mitogenesis, the inventors examined the effect of alternative transcription factors, Pax6 and neurogenin-3 (Ngn3), both of which are implicated in islet development (Schwitzgebel, 2001). β-cell replication was unaffected by overexpression of Pax6 and Ngn3 (FIG. 4C). In summary, forced expression of Pax4 in islets specifically induced DNA synthesis in β-cells recapitulating the effect observed with both activin A and betacellulin.

### Example 4

### Pax4 Overexpression in Islets Induces Genes Implicated in Proliferation

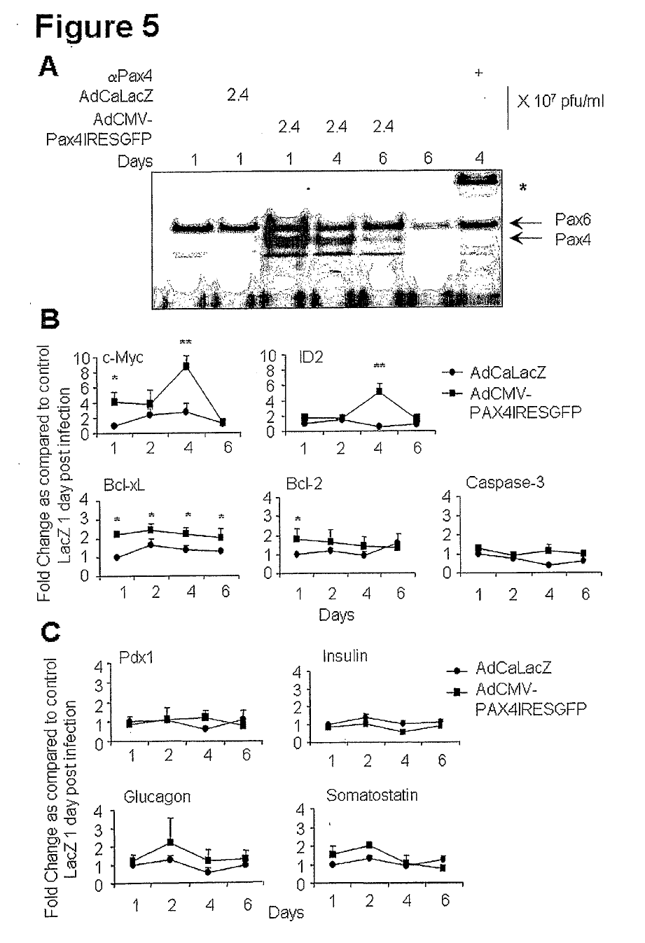

To gain further insight into the potential mechanism by which Pax4 stimulates β-cell replication, the inventors performed a temporal expression profiling of the c-myc proto-oncogene in islets infected for up to six days with either AdCMVPax4IRESGFP or AdCaLacZ. This transcription factor has previously been shown to regulate β-cell mass in mouse islets (Pelengaris et al., 2002). Nuclear protein extracts isolated from AdCMVPax4IRESGFP-transduced islets revealed a transient Pax4 DNA binding activity to the G3 element reaching maximal levels around 1 day post infection (FIG. 5A). Expression levels of c-myc mRNA levels were rapidly induced in AdCMVPax4IRESGFP-transduced islets attaining levels 4-fold higher than those found in control islets 24 h and 48 h after infection (FIG. 5B). These further increased, reaching a maximum of 8-fold above control values at day 4 before returning to basal levels at day 6. Since c-myc stimulates proliferation through activation of the Id2 cell cycle progression regulator, the inventors explored whether this pathway was triggered in Pax4 overexpressing islets (Lasorella et al., 2000). Interestingly, AdCMVPax4IRESGFP-transduced islets expressed Id2 mRNA levels 4-fold higher to those of control islets only 4 days after infection indicating a delayed activation of Id2 gene expression by c-myc (FIG. 5B). Bcl-xL has recently been shown to prevent c-myc-induced islet β-cell apoptosis and to promote proliferation by suppressing the mitochondrial apoptotic pathway (Pelengaris et al., 2002). Consistent with these findings, expression levels of Bcl-xL were found to be 2- to 2.5-fold higher in Pax4-expressing cells while caspase-3 mRNA level remained relatively constant for the 6 day duration of the experiment (FIG. 5B). In addition, Bcl-2 mRNA levels were transiently induced. Taken together, these results suggest that Pax4 stimulates β-cell proliferation through the coordinated activation of the c-myc-Id2 pathway and Bcl-xL gene expression.

### Example 5

### Hormone Expression Profiling of AdCMVPax4IRESGFP-Infected Islets

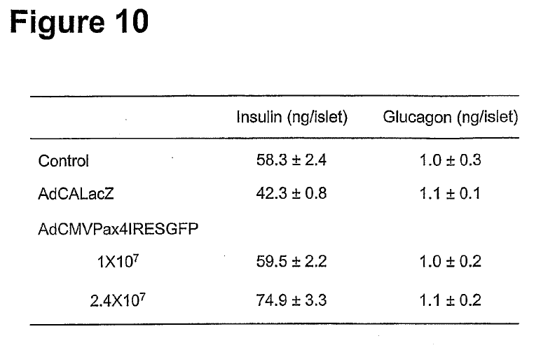

Since Pax4 has been reported to inhibit expression of reporter constructs harbouring either the insulin or glucagon gene promoter in various β and α cell lines (Campbell et al., 1999; Fujitani et al., 1999; Ritz-Laser et at, 2002), the inventors investigated whether endogenous levels of these hormones, as well as somatostatin, were repressed in AdCMVPax4IRESGFP-infected islets by quantitative RT-PCR. In order to evaluate potential time-dependent alterations in gene expression, mRNA levels were measured over a period of 6 days. Surprisingly, insulin, glucagon and somatostatin transcripts remained relatively constant throughout the experiment (FIG. 5C). Consistent with these findings, mRNA levels for the transcription factor Pdx1, which is a major stimulator of insulin and somatostatin gene transcription, also remained stable (FIG. 5C). To confirm the quantitative RT-PCR results, glucagon and insulin protein contents were measured by radioimmunoassay 48 h post infection. Interestingly, a small increase, rather than a decrease, in insulin protein content was measured in islets transduced with the highest concentration of AdCMVPax4IRESGFP while glucagon levels remained stable (FIG. 10, Table 1). Therefore, these results suggest that Pax4 does not function as a transcriptional repressor of insulin and glucagon in mature islet cells.

### Example 6

### Pax4 Transactivates Both the c-myc and Bcl-xL Gene Promoter

To examine whether Pax4 was directly involved in the regulation of Bcl-xL and c-myc transcription, transient transfection assays were performed in BHK-21 cells with luciferase reporter constructs harbouring either gene promoters along with increasing amounts of Pax4. The impact of the type 2 diabetes associated Pax4 mutation R129W, located in the paired DNA binding domain, was also evaluated. To this end, the inventors generated two expression vectors containing either a wild-type (Pax4-myc wt) or mutant Pax4 (Pax4-myc R129W) cDNA fused to the myc epitope. The inventors first validated expression and localization of the proteins encoded by the two constructs in rat insulinoma INS1E cells. Immunofluorescence studies using a myc antibody revealed nuclear localisation of both wild-type and mutant Pax4 in transfected cells (FIG. 6A). In contrast, transfection with a recombinant chimera composed of the vesicular protein; synaptotagmin VII and the c-myc tag resulted in cytoplasmic staining indicating that the tagged peptide does not interfere with proper compartmentalization (FIG. 6A bottom panel). Furthermore, EMSA using equal amounts of in vitro transcribed and translated recombinant proteins (confirmed by Western blotting, data not shown) and the G3 element of the glucagon promoter confirmed the binding activity of the myc-fused wild-type and mutant Pax4 proteins (FIG. 6B, lanes 1 and 3). The specificity of the complexes was demonstrated by supershift assay using the myc antibody (FIG. 6B, lanes 2 and 4). Interestingly, although the recombinant Pax4-myc R129W protein was able to interact with the G3 element, its binding affinity was weaker than the recombinant Pax4-myc wt. Transient transfections in the heterologous BHK-21 cell system revealed that increasing amounts of the Pax4-myc wt expression vector dose dependently stimulated luciferase activity of the c-myc and Bcl-xL gene promoter constructs reaching up to 3.5-fold and 2.7-fold, respectively (FIG. 6C). However, Pax4-myc R129W was less efficient in transactivating both constructs, reaching maximal induction levels of only 2.1-fold and 1.5-fold for the c-myc and Bcl-xL reporter constructs, respectively (FIG. 6C). Transactivation was promoter specific since Pax4 was unable to induce the telomerase promoter Tert-Luc (FIG. 6C). These results clearly demonstrate that Pax4 regulates c-myc and Bcl-xL transcription while the mutant form is less efficient in transactivating both genes.

### Example 7

### Pax4 Overexpression Attenuates Insulin Secretion in Islets

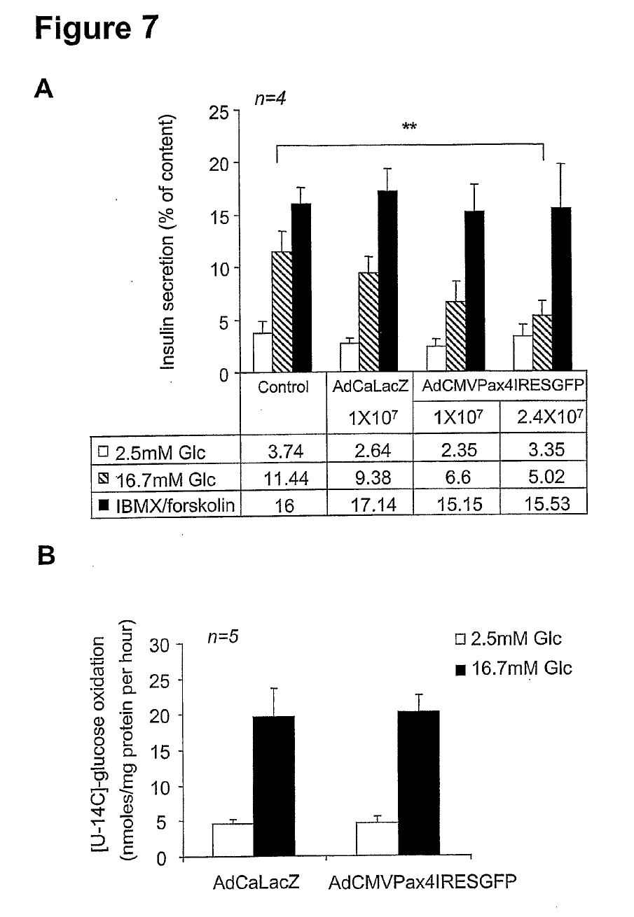

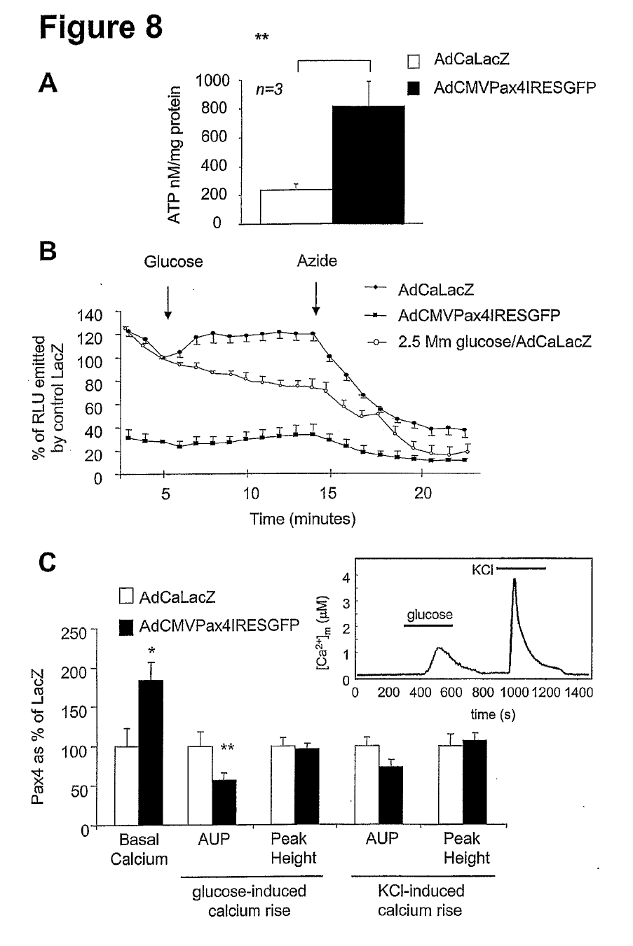

Although increased Bcl-xL expression plays a protective role against c-myc-induced apoptosis, high levels of this mitochondrially-targeted protein were also found to impair insulin secretion (Zhou et al., 2000). The inventors thus examined whether glucose-evoked insulin secretion was altered in Pax4 overexpressing islets. The inventors found that glucose-stimulated insulin secretion was attenuated by 50% in the presence of 2.4×10⁷ pfu/ml of AdCMVPax4IRESGFP 48 h after infection while β-galactosidase-expressing islets and non-infected controls exhibited an expected 3-fold increase in hormone release (FIG. 7A). Interestingly, this level of inhibition corresponds to the percentage of Pax4 infected islet cells. However, inhibition was transient as glucose-induced insulin secretion was restored 6 days post infection at which time Pax4 expression has diminished considerably (data not shown, see also FIG. 5A). Inclusion of 1 μM forskolin/100 μM IBMX, which potentiates the effect of glucose on secretion by rising cAMP levels, restored glucose-induced insulin exocytosis indicating that events downstream of membrane depolarisation are functional in Pax4 expressing cells. The intracellular signalling events that couple metabolism to insulin secretion are triggered by glucose oxidation which stimulates ATP production, closure of K_{ATP} channels, membrane depolarisation, influx of calcium and ultimately exocytosis (Maechler and Wollheim, 2001). To evaluate whether Pax4 curtails this cascade, glucose metabolism as well as ATP levels and mitochondrial calcium concentrations ([Ca²⁺]ₘ) were measured in transfected islets. The rate of glucose oxidation was estimated by measuring the conversion of D-[¹⁴C(U)] to ¹⁴CO₂ in islets. Formation of ¹⁴CO₂ from 15 mM D-[U-¹⁴C] glucose was equally efficient in both control and transduced-islets (4 fold increase, FIG. 7B). However, the inventors found that total cellular ATP levels were 4-fold higher in islets expressing Pax4 as compared to control LacZ islets (FIG. 8A). Total cellular ATP largely reflects sequestered pools in organelles in particular the mitochondria (Detimary et al., 1995). These results prompted us to investigate whether glucose was able to raise cytosolic ATP levels in Pax4 overexpressing islets using targeted expression of the ATP-sensitive bioluminescence probe luciferase (Ishihara et al., 2003). Addition of 16.5 mM glucose to control/LacZ islets resulted in a 23% increase of cytosolic ATP, which was sustained until the injection of azide, a compound that dissipates the mitochondrial membrane potential and thus interrupts ATP formation (FIG. 8B). Cytosolic ATP from islets maintained in 2.5 mM glucose gradually decreased to levels 80% of those at time of glucose injection consistent with low sustained energy consumption. Unexpectedly, basal cytosolic ATP in AdCMVPax4IRESGFP-infected islets was 30% of that measured in control islets. In addition, no significant increase in ATP production was detected in Pax4 islets exposed to 16.5 mM glucose (FIG. 8B). Mitochondrial calcium changes reflect those of the cytosol (Ishihara et al., 2003; Kennedy et al., 1996). Mitochondria targeted-expression of aequorin revealed that resting [Ca²⁺]ₘ was also elevated in the β-cells of Pax4-transduced islets, nearly 2-fold higher than controls (FIG. 8C). High concentrations of extracellular potassium trigger calcium influx across the plasma membrane independently of ATP production. The potassium-induced rise in [Ca²⁺]ₘ was normal in transduced islets, as assessed by both the maximum [Ca²⁺]ₘ response attained (peak height) and the total increase in [Ca²⁺]ₘ (area under the peak; AUP). However, the glucose-induced increase in [Ca²⁺]ₘ (AUP) was blunted in Pax4 expressing islets (40-15% less than controls) although the initial peak response was unchanged. The observed elevation in resting levels of both total cellular ATP and mitochondrial calcium in AdCMVPax4IRESGFP-transduced islets may have caused the attenuated ATP and [Ca²⁺]ₘ responses to glucose. These deficient responses in turn underlie blunted glucose-induced insulin secretion in Pax4 overexpressing islets.

### Example 8

### Pax4 Expression Correlates with Human Islet β-Cell Replication

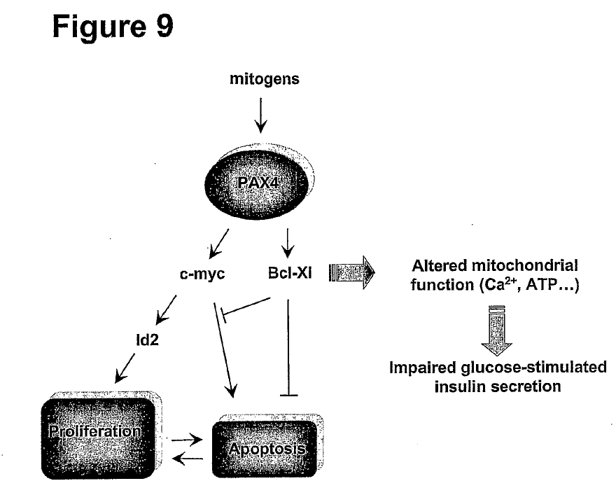

Data presented in examples 2 to 7 suggest that Pax4 is implicated in rat islet β-cell proliferation through the coordinated induction of the c-myc-Id2 replication pathway and expression of the anti-apoptotic Blc-xL gene; see FIG. 9. In this example it was determined that exposure of human isletes to either mitogens or Pax4 overexpression induces β-cell replication.

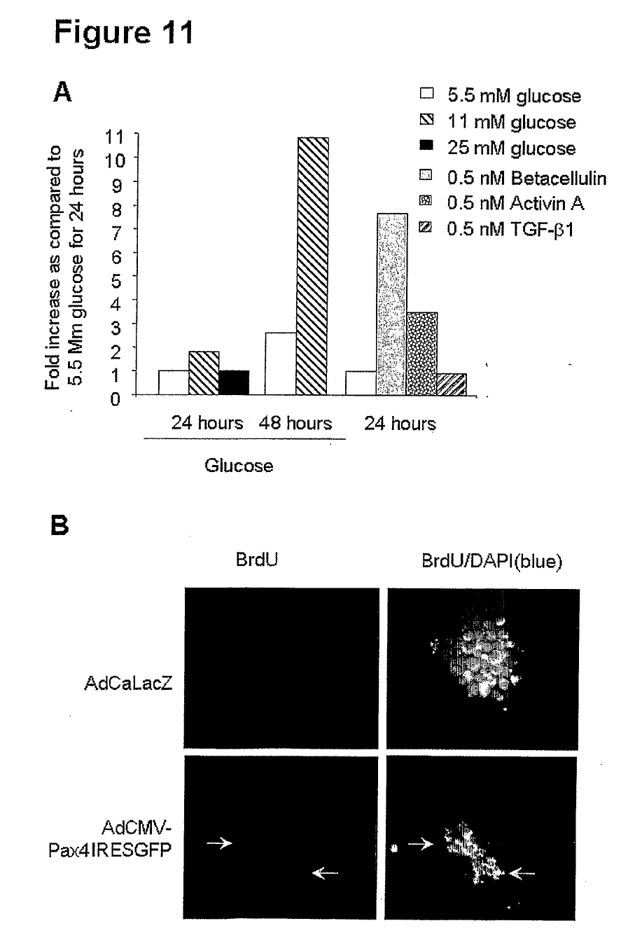

Exposure to 11 mM glucose for 48 hours resulted in an 11-fold increase in Pax4 mRNA levels as compared to control islets cultured in 5.5 mM glucose for 24 hours (FIGS. 11A-B). No significant increase in Pax4 transcript was detected in islets maintained in either 11 or 25 mM glucose for 24 hours. Treatment of islets with either 0.5 nM betacellulin or activin A for 24 hours induced Pax4 mRNA levels by 8 and 4-fold, respectively, as compared to control (FIG. 11A). Pax4 transcript in TGF-β1-treated islets was unaltered. These results suggests that similar to rat islets, Pax4 expression in human islets is induced by factors known to promote β-cell proliferation.

To evaluate the impact of Pax4 on β-cell replication, human islets were infected with an adenoviral construct harbouring the Pax4 cDNA under the control of the CMV promoter (AdCMVPax4IRESGFP). Islets transduced with AdCMVPax4IRESGFP displayed significant BrdU incorporation as compared to control AdCaLacZ-infected islets (FIG. 11B). These results document that human β-cells show the same response pattern in terms of Pax4 expression and mitogenic effect as rat islets.

### Example 9

### Assessment of Pax4 Wild-Type and Loss-Of-Function Mutant in Human Islet β-Cell Replication Using a Novel Adenoviral Inducible System

By using an adenoviral-mediated inducible expression system which allows for the regulated production of Pax4 (ad-mPax4-myc WT in co-infection with Ad-X Tet-On) it could further be confirmed that Pax4 leads to proliferation of adult pancreatic cells.

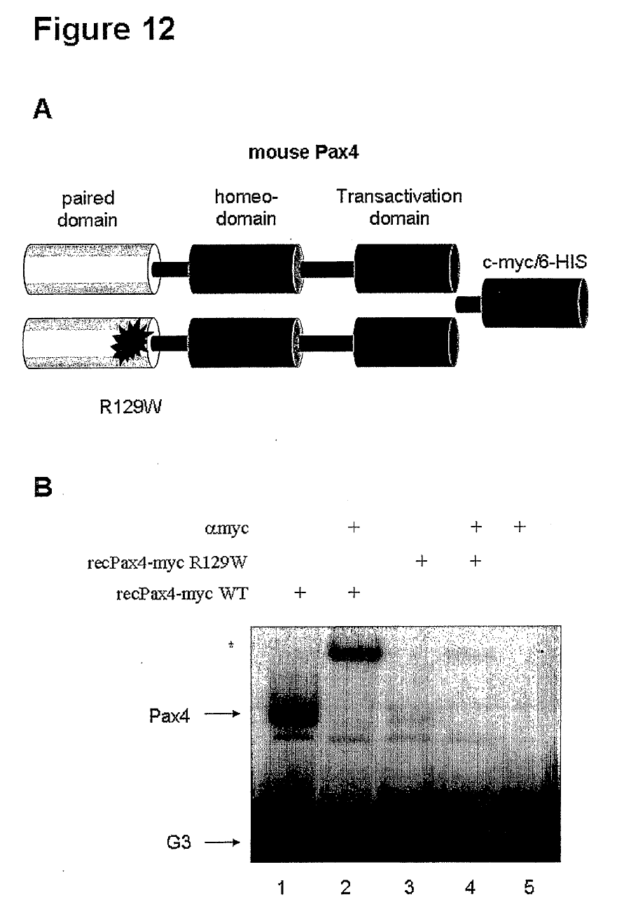

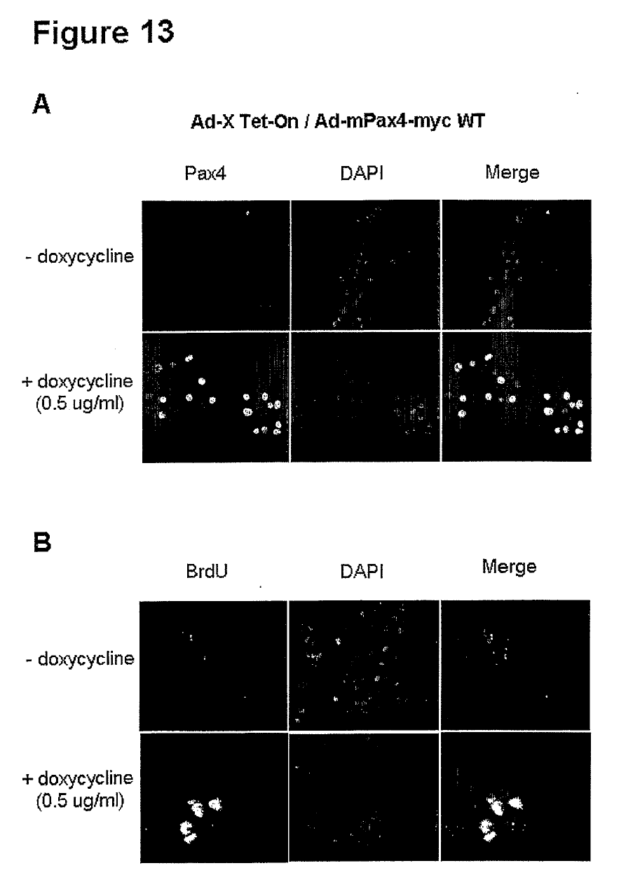

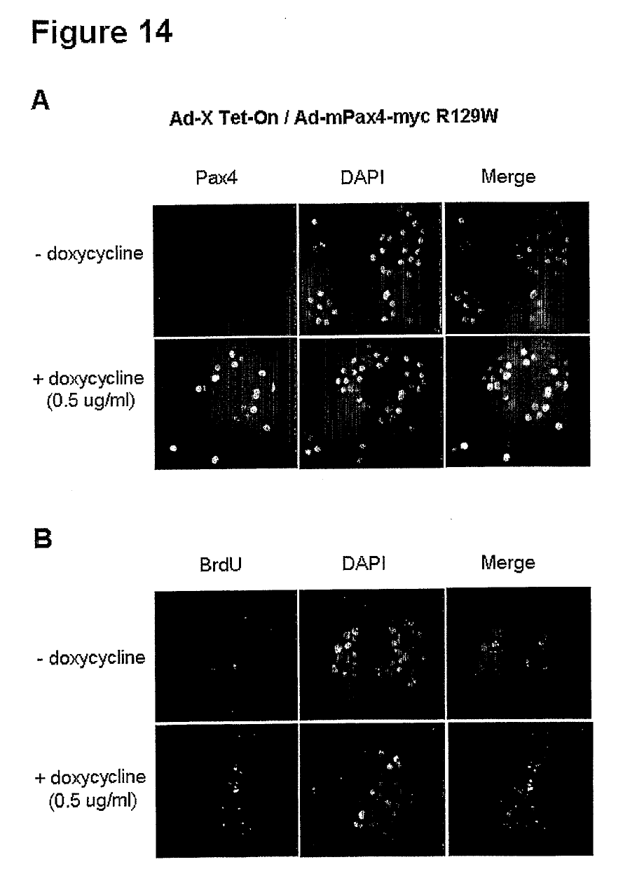

To assess the contribution of Pax4 and its mutant variant on human islet proliferation, the inventors constructed inducible recombinant adenoviruses engineered to express these proteins. In this system, a second adenoviral construct bearing the tet-activator cDNA (Ad-X Tet-On) is co-infected along with the Pax4 recombinant adenoviral constructs (Ad-mPax4-myc WT or Ad-mPax4-myc R129W) in islets. Addition of the inducer, doxycycline (a tetracycline analog), activates the constitutively expressed reverse tet-activator protein (rtTA) that then binds to promoter sequences upstream of the adenoviral Pax4 cDNAs to initiate transcription. In order to detect the proteins after induction by doxycycline, a c-myc/6-HIS tag was integrated at the carboxy terminal end of the two polypeptides; see also example 6. Addition of the c-myc epitope antibody resulted in the supershift of both complexes (FIG. 12B) indicating functional fusion proteins. Primary human islets were then transduced with either Ad-mPax4-myc WT or Ad-mPax4-myc R129W along with Ad-X Tet-On at the optimal ratio of 2:1. In the absence of doxycycline, the immunoreactive c-myc epitope could not be detected in islet cells (FIGS. 13A and 14A). However, addition of doxycycline, resulted in the induction of either mPax4-myc WT or R129W in the nuclei of approximately 70% of islets cells (FIGS. 13B and 14B). Concomitant with doxycycline-induced mPax4 WT expression, approximately 10% of cells incorporated BrdU indicative of cell proliferation (FIG. 13B). In contrast, stimulation of cell replication as assessed by BrdU could not be detected in cells expressing mPax4-myc R129W (FIG. 14B). Importantly, in the absence of doyxycline there was no visible β-cell proliferation (FIGS. 13A-B and 14A-B). These results clearly indicate that Pax4 is an important factor involved in islet cell replication and that the R129W mutation abrogates the function of the transcription factor.

### Example 10

### Pax4 Protects Human Islets from Cell Death

As documented above, stimulation of the transcription factor Pax4, either by mitogens or forced expression, promotes rat and human islet β-cell proliferation through c-myc activation while potentially protecting from apoptosis through Bcl-xL gene expression. The impact of Pax4 and its loss-of-function mutant on human islet survival using the adenoviral-mediated inducible expression system described above is assess in this example.

Freshly isolated human islets, obtained from the Cell Isolation and Transplantation Laboratory in Geneva, were maintained for 48 hours in CMRL-1066 medium supplemented with 10% FCS, 100 Units/ml penicillin, 100 μg/ml streptomycin and 100 μg/ml gentamycin. Partially trypsinized islets were then infected with either Ad-mPax4-myc WT or Ad-mPax4-myc R129W (2.4×10⁷ pfu/ml) along with the adenoviral construct harbouring the tetracycline transcriptional activator (Ad-X Tet-On, 1.2×10⁷ pfu/ml). Cells were rinsed 90 minutes post infection and replenished with fresh media supplemented with or without doxycycline (0.25 and 0.5 μg/ml) for 24 h post infection. Cells were then incubated for 24 h in the presence of IFN-γ, IL-1β and TNF-α, (2 ng/ml each) to induce apoptosis. Cell death was measured by direct TUNEL (terminal deoxynucleotidyl transferase (TdT)-mediated dUTP nick end labelling) assay. Briefly, DNA strand breaks (free 3′OH ends in genomic DNA) caused by cytokine-induced apoptosis were labelled with fluorescein-dUTP using an optimised TdT. Incorporated fluorescein-dUTP in apoptotic cells was then detected and quantified directly under a fluorescence microscope.

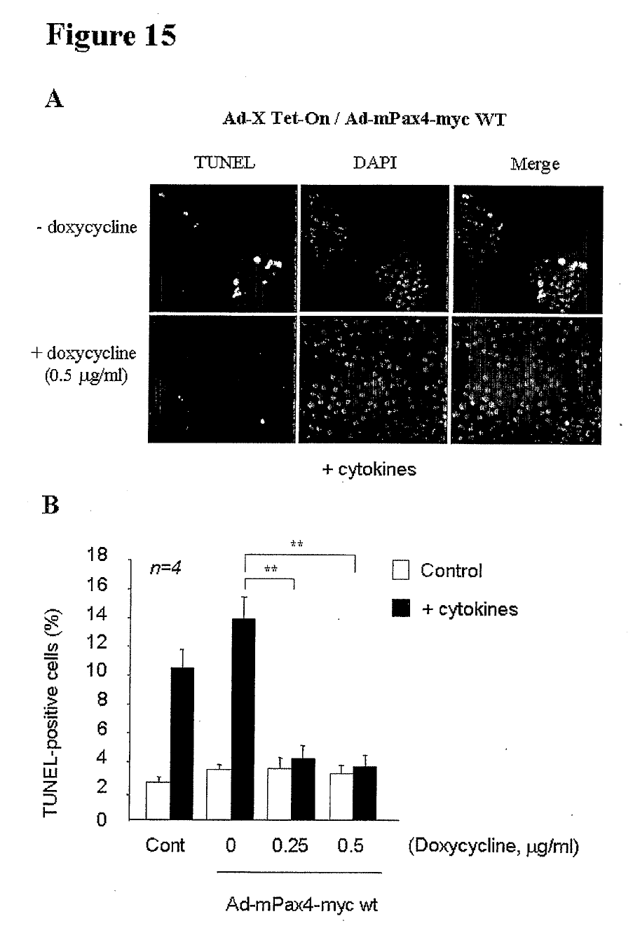

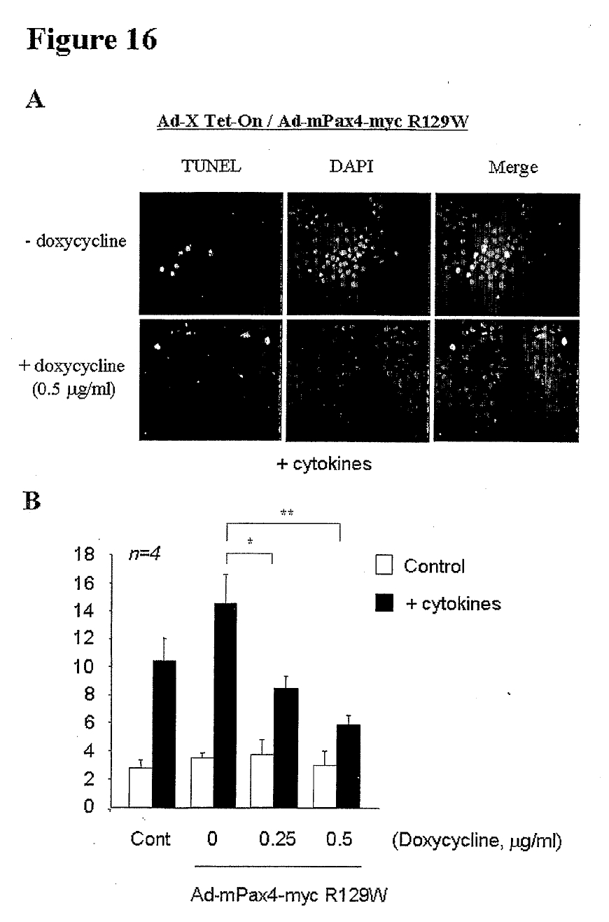

As shown above, Pax4 is an important factor involved in islet cell replication and an mutation, namely the R129W mutation abrogates the function of the transcription factor. In this experiment transduced human islets were exposed to a cocktail of cytokines to induce apoptosis. Adenoviral-infected islets in the absence of both doxycycline and cytokines exhibited a 1.3 fold increase in apoptosis as compared to control non-infected islets (FIGS. 15 and 16, B). Treatment with cytokines provoked a further 4.4 fold increase in cell death. In contrast, Pax4 wt expression (induced by the two concentrations of doxycycline) conferred complete protection against apoptosis (FIGS. 15 A and B). The R129W mutation showed an attenuate protection against cytokine-induced cell death (FIGS. 16 A and B).

Accordingly, Pax4 promotes survival in human islets. With the help of doxycycline inducible adenoviral vectors it was shown that the wild type Pax4 upon drug stimulation shielded human islet cells from cytokine-induced apoptosis while the mutant was less efficient. Furthermore, Pax4 levels were maintained close to physiological ranges indicating a specific effect of the transcription factor on survival.

These results show that Pax4 promotes human islet cell replication, and, most importantly also confers survival through the activation of Bcl-xL. The diabetes-linked mutant form of Pax4 attenuates these cellular processes. It is of note that affected subjects suffer from β-cell death.

Three major conclusions arise as a result of our studies: 1) an adenoviral system has been developed that allows doxycycline-dependent expression of either wild-type or mutant Pax4 in primary human islets; 2) inducible expression of wild-type Pax4 stimulated proliferation while 3) induction of mutant R129W variant had no effect on cell division. These results strongly indicate that Pax4 is a key regulator of islet cell proliferation and that loss-of-function mutants impair β-cell replenishment. Such mutations may lead to reduced β-cell mass and thus could be causally related to the development of both type 1 and type 2 diabetes. A practical consequence of our findings would be to expand the β-cell mass in vitro by the regulated expression of Pax4 prior to islet transplantation for the treatment of diabetic patients. The controlled and transient expression of Pax4 avoids prolonged expression of a mitogenic protein that may have untoward effects on islet function.

### Example 11

### RNA Interference of Pax4 Reveals a Direct Contribution of the Transcription Factor on Survival of Insulin-Producing Cells

As discussed above, the paired and homeo domain-containing transcription factor Pax4 plays an important role in β-cell development during embryogenesis while its function in mature islets is still controversial (Sosa-Pineda, 2004, Mol Cells 18, 289-94). Polymorphisms as well as mutations in this gene have been associated with both type 1 and type 2 diabetes suggesting a fundamental role of Pax4 in islet function (Biason-Lauber, 2005, Diabetologia 48, 900-905; Holm, 2004, Diabetes 53, 1584-91; Mauvais-Jarvis, 2004, Hum. Mol. Genet. 13, 3151-3159; Shimajiri, 2003, Biochem. Biophys. Res. Commun. 282, 34-40). Herein above, it was demonstrated that increased Pax4 expression, either by mitogens or forced expression, promotes β-cell proliferation in mature rat islets by simultaneously inducing the c-myc-Id2 pathway and the anti-apoptotic gene Bcl-xL. The impact of Pax4 and its diabetes linked variant R129W on human islet proliferation and survival was also assessed using novel doxycycline inducible recombinant adenoviruses engineered to express these proteins tagged to the myc epitope (Ad-mPax4-myc wt or Ad-mPax4-myc R129W). Induction of Pax4 wt expression stimulated β-cell replication and conferred complete protection against cytokine-induced apoptosis. In contrast, the R129W mutation showed an attenuated protection against cell death. Thus Pax4 promotes rat and human islet cell replication as well as conferring survival potentially through the activation of Bcl-xL. The diabetes-linked mutant form of Pax4 is incapable of reproducing these effects.

Furthermore, the impact of the mitogens activin A and betacellulin on endogenous Pax4 gene expression in human islets at high and low glucose concentrations was evaluated. In addition, a RNA interference (RNAi) strategy to suppress Pax4 in order to evaluate the direct contribution of the transcription factor on β-cell plasticity.

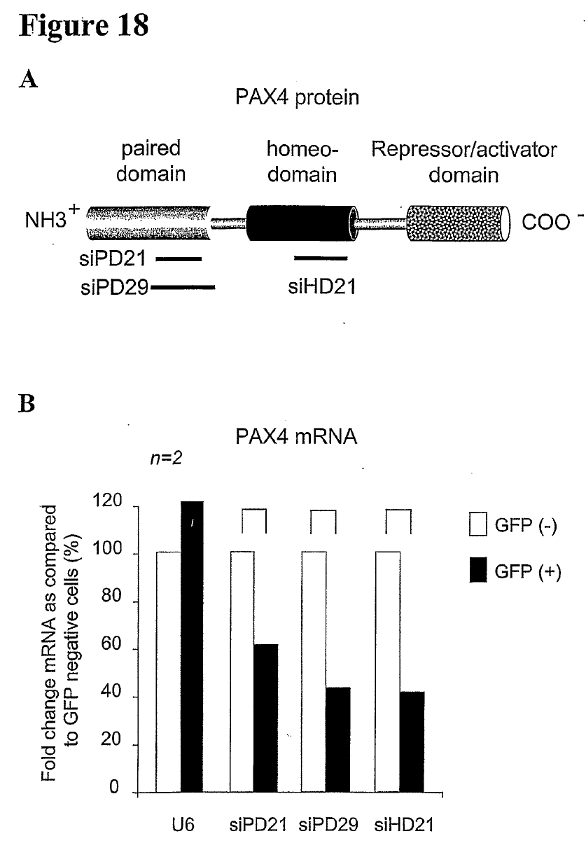

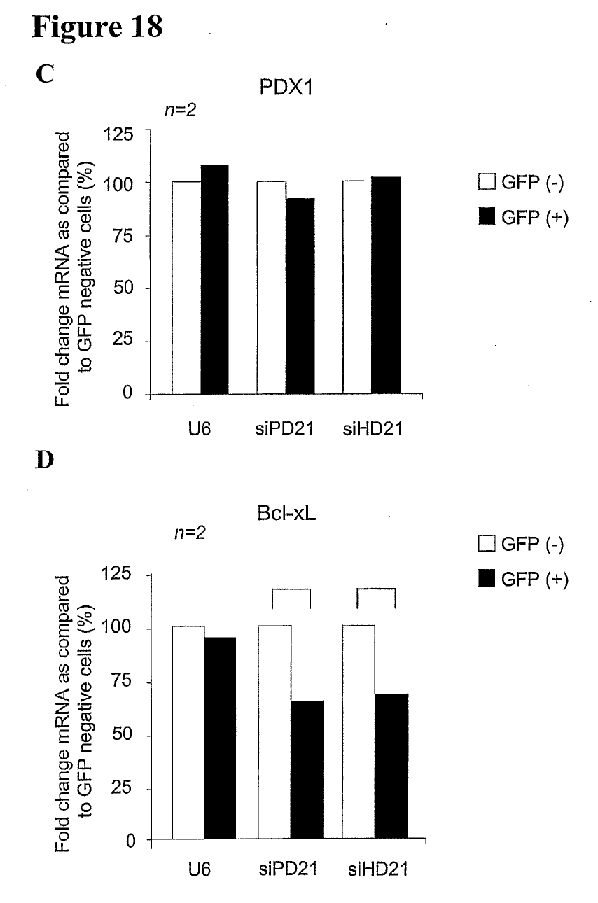

For this experiment, isolated human islets were exposed to 5.5 or 11 mM glucose complemented with 0.5 nM of activin A, betacellulin or TGF-β 1 for 24 hours. Steady state mRNA levels for Pax4 were quantified by real-time RT-PCR and normalized to cyclophilin. Two 21-nucleotide Pax4 hairpin RNA structures targeted to either the paired domain (siPD21; 5′-GCA GGC AAG AGA AGC TGA AAT-3′; SEQ ID NO: 21) or the homeodomain (siHD21; 5′-GGC TCG AAT TGC CCA GCT AAA-3′; SEQ ID NO: 22) of Pax4 were cloned into the pDLDU6 vector (Gauthier, 2003, Diabetologia 46, A47) (see FIG. 18A). An extended siRNA of 29 nucleotides targeted to the same sequence of the paired domain was also generated and cloned into pDLDU6 (siPD29; 5′-GGC TCG AAT TGC CCA GCT AAA GGA TGA GT-3; SEQ ID NO: 23). Constructs were transfected into the rat insulinoma cell line INS-1E along with GFP using lipofectamine. Subsequent to cell sorting using GFP (72 hours post-transfection), RNA was isolated and the effect of siPD21, siPD29 and siHD21 on endogenous Pax4, Pdx1 and Bcl-xL transcript levels was quantified by real time RT-PCR. Alternatively, 48 hours post transfection, cells were incubated with a cocktail of cytokines (1 ng/ml of each Il-1, TNFα and INFγ) for an additional 24 hours and cell death was estimated using the TUNEL assay (Roche, CH).

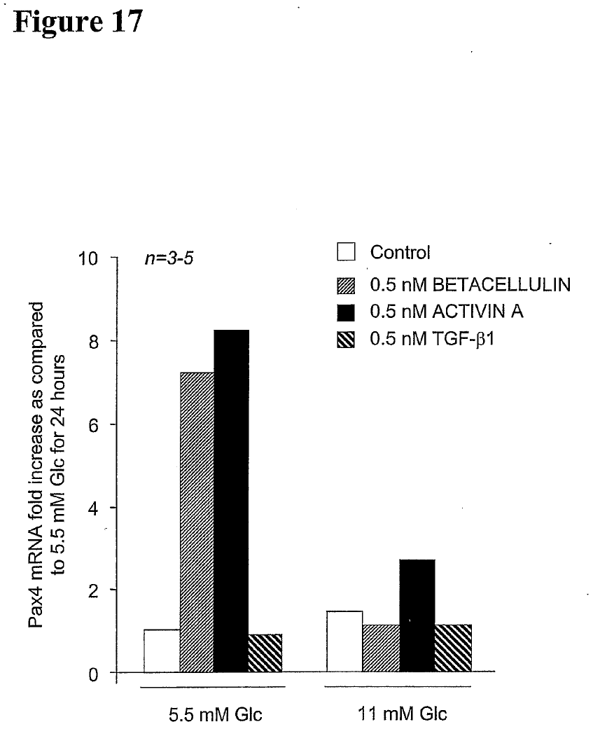

Low but consistent Pax4 mRNA levels were detected in isolated human islets. Treatment with either 0.5 nM activin A or betacellulin in the presence of 5.5 mM glucose resulted in a 7- and 8-fold increase in the Pax4 transcript, respectively (FIG. 17). In contrast, TGF-β 1 was ineffective. Interestingly, 11 mM glucose did not stimulate Pax4 gene expression and abrogated the effect of betacellulin and activin A (FIG. 17). These results provide evidence that Pax4 activity is regulated by physiological stimuli in human islets.

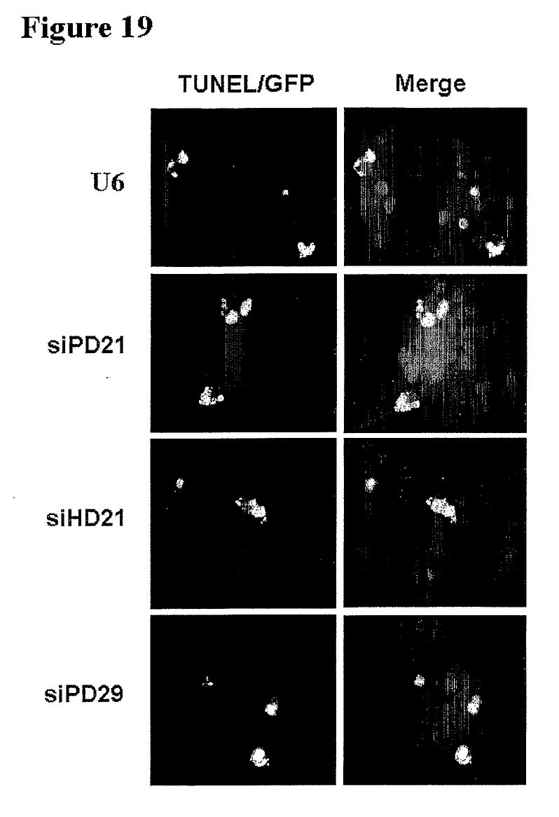

However, glucose would appear to have an inhibitory effect on activin A and betacellulin-mediated induction of Pax4 gene expression in human islets. It was next evaluated the impact of Pax4 on β-cell survival by RNAi. The transformed INS-1E cell line was employed. Said cell line is characterized by uncontrolled cellular proliferation while retaining high levels of insulin and exhibiting normal stimulus-secretion coupling (Asfari, 1992, Endocrinology 130, 167-78; Merglen, 2004, Endocrinology 148, 667-78). Like human insulinomas (Miyamoto, 2001, Biochem. Biophys. Res. Commun. 282, 34-40), INS-1E cells express high levels of Pax4 mRNA and provide a useful tool to investigate the impact of the transcription factor on cell proliferation and survival. Pax4 steady state mRNA levels were lowered by 40, 55 and 60% in INS-1E cells co-expressing siPD21, siPD29 or siHD21, respectively along with GFP (FIG. 18B). A 29-mer was more proficient in suppressing the Pax4 transcript as compared to a 21-mer directed against the same sequence within the paired domain of Pax4 (Siolas, 2005, Nat. Biotechnol. 23, 227-31). Repression was specific since mRNA levels for Pdx1 remained constant in cells expressing either siPD21 or siHD21 (FIG. 18C). It was also monitored STAT1 mRNA levels, which increase in response to induction of the interferon system (Sledz, 2003, Nat. Cell Biol. 5, 834-9). Similarly to Pdx1, STAT1 transcripts were unaltered indicating that the RNAi did not provoke an interferon response (data not shown). In contrast, mRNA levels for the Pax4 target gene Bcl-xL were suppressed by 40% using either siPD21 or siHD21 (FIG. 18D). Inhibition of the anti-apoptotic gene did not promote INS-1E cell death under control conditions (data not shown). However, when challenged with a cocktail of cytokines, cells co-expressing phogrin-GFP along with siPD21, siPD29 or siHD21 displayed increased TUNEL as compared to INS-1E co-transfected with phogrin-GFP and U6 (FIG. 19). Taken together, these results clearly indicate that suppression of Pax4 results in decreased Bcl-xL mRNA levels rendering INS-1E cells more susceptible to cytokine-induced apoptosis.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### X. Further References

The following references, to the extent that they provide exemplary procedural or other details supplementary to those set forth herein, are specifically incorporated herein by reference.
U.S. Pat. No. 4,683,202
U.S. Pat. No. 4,797,368
U.S. Pat. No. 4,892,538
U.S. Pat. No. 5,139,941
U.S. Pat. No. 5,626,561
U.S. Pat. No. 5,787,900
U.S. Pat. No. 5,843,069
U.S. Pat. No. 5,928,944
U.S. Pat. No. 6,013,638
U.S. Pat. No. 6,071,697
U.S. Pat. No. 6,099,831
U.S. Pat. No. 4,682,195
U.S. Pat. No. 5,011,472
Altman et al., Diabetes, 35(6):625-633, 1986.
Altschul, J. Mol. Biol., 215:403-410, 1990.
Altschul, J. Mol. Evol., 36:290-300, 1993.
Altschul, Nucl. Acids Res., 25:3389-3402, 1997.
Ausubel et al., In: Current Protocols in Molecular Biology, John, Wiley & Sons, Inc, New York, 1996.
Baichwal and Sugden, In: Gene Transfer, Kucherlapati (Ed.), NY, Plenum Press, 117-148, 1986.
Bauer et al., Blood, 86(6):2379-2387, 1995.
Becker et al., Meth. Cell. Biol., 43:161-189, 1994.
Becker et al., Methods Cell Biol., 43(A):161-189, 1994.
Becker et al., J. Biol. Chem., 269(33):21234-212338, 1994a.
Becker et al., J. Biol. Chem., 271(1):390-394, 1996.
Becker et al., Methods Cell Biol., 43(A):161-189, 1994b.
Bell, Nature, 414:788-791, 2001.
Bestwick et al., Proc. Natl. Acad. Sci. USA, 85(15):5404-5408, 1988.
Blysczuk, PNAS, 100:908-1003, 2003.
Bonner-Weir, Endocrinology, 141:1926-1929, 2000.
Bonner-Weir, Trends Endocrinol Metab, 11:375-378, 2000.
Brandle, Diabetes Care, 24:1253-1258, 2001.
Brink, Mech. Dev., 100:37-43, 2001.
Brutlag, Comp. App. Biosci., 6:237-245, 1990.
Buteau, Diabetes, 52:124-132, 2003.
Butler, Diabetes, 52:102-110, 2003.
Campell, FEBS, 463:53-57, 1999.
Challet, J. Biol. Chem., 276:3791-3797, 2001.
Chan, Diabetes, 48:997-1005, 1999.
Chang et al., Hepatology, 14:134A, 1991.
Chen and Okayama, Mol. Cell. Biol., 7(8):2745-2752, 1987.
Clark et al., Hum. Gene Ther., 6(10):1329-1341, 1995.
Clark et al., Am. J. Clin. Pathol., 93(1):58-69, 1990.
Coffin, In: Virology, Fields et al. (Eds.), Raven Press, NY, 1437-1500, 1990.
Collins, Biotechniques, 24:803-808, 1998.
Collombat, Genes Dev., 17:2591-2603, 2003.
Cotten et al., Proc. Natl. Acad. Sci. USA, 89(13):6094-6098, 1992.
Couch et al., Am. Rev. Resp. Dis., 88:394-403, 1963.
Coupar et al., Gene, 68:1-10, 1988.
Curiel, Nat. Immun., 13(2-3):141-164, 1994.
Davis, Cancer Res., 54:2869-2872, 1994.
Demeterco, J. Clin. Endocrinol. Metab., 85:3892-3896, 2000.
Detimary, J. Clin. Invest., 96:1738-1745, 1995.
Dohrmann, Mech. Dev., 92:47-54, 2000.
Dor, Nature, 429:41-46, 2004.
Drucker, Endocrinology, 142:521-527, 2001.
Dunbar, Int. J. Biochem. Cell Biol., 32:805-815, 2000.
Dupont, Diabetologica, 42:480-484, 1999.
Efrat et al., Proc. Natl. Acad. Sci. USA, 85(23):9037-9041, 1988.
EPO 0273085
Fechheimer, et al., Proc Natl. Acad. Sci. USA, 84:8463-8467, 1987.
Ferkol et al., FASEB J., 7:1081-1091, 1993.
Flotte and Carter, Gene Ther., 2(6):357-62, 1995.
Flotte et al., Am. J. Respir. Cell Mol. Biol., 7(3):349-356, 1992.
Flotte et al., Proc. Natl. Acad. Sci. USA, 90(22):10613-10617, 1993.
Fournier, Transplant Proc., 28:2866-2868, 1996.
Fraley et al., Proc. Natl. Acad. Sci. USA, 76:3348-3352, 1979.
Friedmann, Science, 244:1275-1281, 1989.
Fritschy et al. Diabetes, 40(1):37-43, 1991.
Froguel, N. Engl. J. Med., 328:697-702, 1993.
Fujitani, Mol. Cell Biol., 19:8281-8291, 1999.
Gainer et al., Transplantation, 61(11):1567-1571, 1996.
Galili, Nat. Genet., 5:230-235, 1993.
Gallichan et al., Hum. Gene Ther., 9(18):2717-2726, 1998.
Gauthier, Atherosclerosis, 142:301-307, 1999b.
Gauthier, J. Lipid Res., 40:1284-1293, 1999a.
Gauthier, Mol. Endocrinol., 16:170-183, 2002.
German et al., Mol. Cell. Biol., 12(4):1777-1788, 1992.
German et al., 1990
Ghosh and Bachhawat, In: Liver Diseases, Targeted Diagnosis and Therapy Using Specific Receptors and Ligands, Wu et al. (Eds.), Marcel Dekker, NY, 87-104, 1991.
Ghosh-Choudhury et al., EMBO J., 6:1733-1739, 1987.
Gittes, Proc. Natl. Acad. Sci. USA, 89:1128-1132, 1992.
Gomez-Foix et al., J. Biol. Chem., 267:25129-25134, 1992.
Gopal, Mol. Cell. Biol., 5:1188-1190, 1985.
Graham and Prevec, In: Methods in Molecular Biology: Gene Transfer and Expression Protocol, Murray (Ed.), Humana Press, Clifton, N.J., 7:109-128, 1991.
Graham and Van Der Eb, Virology, 52:456-467, 1973.
Graham et al, J. General Virology, 36:59-74, 1977.
Grunhaus and Horwitz, Seminar in Virology, 3:237-252, 1992.
Hagenfeldt-Johansson, Endocrinology, 142:5311-5320, 2001.
Hansen, J. Clin. Endocrinol. Metab., 85:323-1326, 2000.
Harland and Weintraub, J. Cell Biol.,101(3):1094-1099, 1985.
Hellman, Ann. NY Acad. Sci., 131:541-558, 1965.
Heremans, J. Cell Biol., 159:303-312, 2002.
Hermonat and Muzycska, Proc. Natl. Acad. Sci. USA, 81:6466-6470, 1984.
Hersdorffer et al., DNA Cell Biol., 9:713-723, 1990.
Herz and Gerard, Proc. Natl. Acad. Sci. USA, 90:2812-2816, 1993.
Higgins and Hames, In: Nucleic acid hybridization, a practical approach, IRL Press Oxford, Washington D.C., 1985.
Holm et al., Diabetes, 53:1584-1591, 2004.
Horwich et al. J. Virol., 64:642-650, 1990.
Ishihara, J. Clin. Invest., 104:1621-1629, 1999.
Ishihara, Nat. Cell Biol., 5:330-335, 2003.
Janjic, Pancreas, 13:166-172, 1996.
Jones and Shenk, Cell, 13:181-188, 1978.
Kafri et al., J. Virol., 73(1):576-584, 1999.
Kahan, Diabetes, 52:2016-2024, 2003.
Kanatsuka, Metabolism, 51:1161-1165, 2002.
Kaneda et al., Science, 243:375-378, 1989.
Kaplitt et al., Nat. Genet., 8(2):148-154, 1994.
Karlsson et al., EMBO J., 5:2377-2385, 1986.
Kashyap, Diabetes, 52:2461-2474, 2003.
Kato et al, J. Biol. Chem., 266:3361-3364, 1991.
Kelleher and Vos, Biotechniques, 17(6):1110-7, 1994.
Kennedy, J. Clin. Invest., 98:2524-2538, 1996.
Kiem et al., Blood, 83(6):1467-1473, 1994.
Klein et al., Nature, 327:70-73, 1987.
Kloppel, Surv. Synth. Pathol. Res., 4:110-125, 1985.
Kojima, Nat. Med., 9:596-603, 2003.
Kotin et al., Proc. Natl. Acad. Sci. USA, 87(6):2211-2215, 1990.
Kozmik, Proc. Natl. Acad. Sci. USA, 92:5709-5713. 1995.
Kristinsson, Diabetologia, 44:2098-2103, 2001.
Lacy et al., Science, 254(5039):1782-1784, 1991.
LaFace et al., Virology, 162(2):483-486, 1988.
Lasorella, Nature, 407:592-598, 2000.
Laughlin et al., J. Virol., 60(2):515-524, 1986.
Le Gal La Salle et al., Science, 259:988-990, 1993.
Lebkowski et al., Mol. Cell. Biol., 8(10):3988-3996, 1988.
Levrero et al., Gene, 101:195-202, 1991.
Liang et al., 1996
Luo et al., Proc. Natl. Acad. Sci. USA, 93:8907-8912, 1996.
Maassen, Exp. Clin. Endocrinol. Diabetes, 109:127-134, 2001.
Madsen et al., Proc. Natl. Acad. Sci. USA, 85(18):6652-6656, 1988.
Maechler, Nature, 402:685-689, 1999.
Maechler, Nature, 414:807-812, 2001.
Maedler, Diabetes, 50:1683-1690, 2001.
Malecki, Nature Genet., 23:323-328, 1999.
Mann et al., Cell, 33:153-159, 1983.
Margue, Oncogene, 19:2921-2929, 2000.
Markowitz et al., J. Virol., 62:1120-1124, 1988.
McCarty et al., J. Virol., 65(6):2936-2945, 1991.
McLaughlin et al., J. Virol., 62(6):1963-1973, 1988.
Melloul et al., Proc. Natl. Acad. Set. USA, 90(9):3865-3869, 1993.
Merglen, Endocrinology, 145:667-678, 2004.
Michael, Mol. Cell, 6:87-97, 2000.
Miller and Rosman, Biotechniques, 7(9):980-982, 984-986, 989-990, 1989.
Miller et al., Am. J. Clin. Oncol., 15(3):216-221, 1992.
Miller et al., J. Pharmacol. Exp. Ther., 264:11-16, 1993.
Mitchell, Mol. Genet. Metab., 77:35-43, 2002.
Miyamoto, Biochem. Biophys. Res. Commun., 282:34-40, 2001.
Miyoshi et al., J. Virol., 72(10):8150-8157, 1998.
Muratovska, Oncogene, 22:6045-6053, 2003.
Muzyczka, Curr. Topics Microbiol. Immunol., 158:97-129, 1992.
Nicolas and Rubenstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Ohi et al., Gene, 89(2):279-282, 1990.
Osborne et al., Hum. Gene Ther., 1(1):31-41, 1990.
O'Shea and Sun, Diabetes, 35(8):943-946, 1986.
Paris, Endocrinology, 144:2717-2727, 2003.
Paskind et al., Virology, 67:242-248, 1975.
PCT Appln. PCT/US99/00553
PCT Appln. WO 89/01967
PCT Appln. WO 90/02580
PCT Appln. WO 90/15637
PCT Appln. WO 91/09939
PCT Appln. WO 91/10425
PCT Appln. WO 91/10470
PCT Appln. WO 98/29566
PCT Appln. WO95/29989
Pelengaris, Cell, 109:321-334, 2002.
Perales et al., Proc. Natl. Acad. Sci. USA, 91:4086-4090, 1994.
Potter et al., Proc. Natl. Acad. Sci. USA, 81:7161-7165, 1984.
Racher et al., Biotechnology Techniques, 9:169-174, 1995.
Ragot et al., Nature, 361:647-650, 1993.
Renan, Radiother. Oncol., 19:197-218, 1990.
Rich et al., Hum. Gene Ther., 4:461-476, 1993.
Ridgeway, In: Vectors: A survey of molecular cloning vectors and their uses, Rodriguez and Denhardt (Eds.), Stoneham:Butterworth, 467-492, 1998.
Rippe et al., Mol. Cell. Biol., 10:689-695, 1990.
Ritz-Laser, Diabetologia, 45:97-107, 2002.
Rosenfeld et al., Science, 252:431-434, 1991.
Rosenfeld, et al., Cell, 68:143-155, 1992.
Roux et al., Proc. Natl. Acad. Sci. USA, 86:9079-9083, 1989.
Ryan, Diabetes, 50:710-719, 2001.
Ryan, Diabetes, 51:2148-2157, 2002.
Ryffel, J. Mol. Endocrinol., 27:11-29, 2001.
Salehi, J. Physiol., 15:579-591, 1999.
Saltiel, Nature, 414:99-806, 2001.
Sambrook et al., In: Molecular cloning, Cold Spring Harbor Laboratory Press, Cold
Spring Harbor, N.Y., 2001.
Samulski et al., EMBO J., 10:3941-3950, 1991.
Samulski et al., J. Virol, 63:3822-3828, 1989.
Samulski et al., J. Viral, 63:3822-3828, 1989.
Sato et al., Proc. Natl. Acad. Sci. U.S.A. 48:1184-1190, 1962.
Schreiber, EMBO J., 7:4221-4229, 1988.
Schwitzgebel, Mol. Cell. Endocrinol., 185:99-108, 2001.
Shapiro, N. Engl. J. Med., 343:230-238, 2000.
Shelling and Smith, Gene Therapy, 1:165-169, 1994.
Shih, Diabetes, 50:2472-2480, 2001.
Shimajiri, Biochem. Biophys. Res. Commun., 302:342-344, 2003.
Shimajiri, Diabetes, 50:2864-2869, 2001.
Smith, J. Biol. Chem., 275:36910-36919, 2000.
Smith, Mol. Endocrinol., 18:142-149, 2004.
Soldevila et al. J. Autoimmun., 4(2):291-306, 1991.
Sosa-Pineda, Nature, 386:399-402, 1997.
Stockschlaeder et al., Hum. Gene Ther., 2(1):33-39, 1991.
Stoffers, J. Clin. Invest., 102:232-241, 1998.
Stratford-Perricaudet and Perricaudet, In: Human Gene Transfer, Eds, Cohen-Haguenauer and Boiron, John Libbey Eurotext, France,
Stratford-Perricaudet et al., Hum. Gene. Ther., 1:241-256, 1990.
Street, Int. J. Biochem. Cell Biol., 36:667-683, 2004.
Sullivan et al., J. Infect. Dis., 164(4):781-784, 1991.
Tao, Diabetes, 47:1650-1653, 1998.
Temin, In: Gene Transfer, Kucherlapati (Ed.), NY, Plenum Press, 149-188, 1986.
Thompson, Nucl. Acids Res., 2:4673-4680, 1994.
Top et al, J. Infect. Dis., 124:155-160, 1971.
Tratschin et al., Mol. Cell. Biol., 4:2072-2081, 1984.
Tratschin et al., Mol. Cell. Biol., 5:3258-3260, 1985.
Tur-Kaspa et al., Mol. Cell. Biol., 6:716-718, 1986.
Ueda, FEBS Lett., 480:101-105, 2000.
Ueda, J. Virol., 70:4714-472, 1996.
Van der Laan et al., Nature, 407:90-94, 2000.
Wagner et al., Proc. Natl. Acad. Sci. USA 87(9):3410-3414, 1990.
Walsh et al., J. Clin. Invest, 94:1440-1448, 1994.
Walsh et al., J. Clin. Invest, 94:1440-1448, 1994.
Wang, Dev. Biol., 266:178-189, 2400.
Wei et al., Gene Therapy, 1:261-268, 1994.
Weir, Semin. Cell. Dev. Biol., 15:347-357, 2004.
Weng, Diabetologia, 44:249-258, 2001.
Wong et al., Gene, 10:87-94, 1980.
Wu and Wu, Adv. Drug Delivery Rev., 12:159-167, 1993.
Wu and Wu, Biochemistry, 27:887-892, 1988.
Wu and Wu, J. Biol. Chem., 262:4429-4432, 1987.
Xu, Diabetes, 48:2270-2276, 1999.
Xu, Mol. Cell. Endocrinol., 170:79-89, 2000.
Yamagata, Diabetes, 51:114-123, 2002.
Yang et al., J. Virol., 68:4847-4856, 1994.
Yang et al., Proc. Natl. Acad. Sci. USA, 87:9568-9572, 1990.
Yoder et al., Blood, 82 (Supp.): 1:347A, 1994.
Zalzman, Proc. Natl. Acad. Sci. USA, 100:2426-2431, 2003.
Zhang, Diabetes, 50(1): S10-14, 2001.
Zhou et al., Exp. Hematol, 21:928-933, 1993.
Zhou et al., J. Exp. Med., 179:1867-1875, 1994.
Zhou, Am. J. Physiol. Endocrinol. Metab., 278:E340-351, 2000.
Zimmet, Nature, 414:782-787, 2001.

1. An in vitro method for the generation and purification of pancreatic β-cells, comprising the steps of:
(a) providing an adult cell derived from a mammalian pancreatic islets or an explant culture of an adult pancreatic islets with functional wild-type Pax4; and
(b) detecting and isolating, from said adult cell or explant culture, β-cells that proliferate in response to the contact with Pax4.

2. The method of claim 1, wherein said functional wild-type Pax4 is administered to said cell or explant culture as a nucleic acid molecule.

3. The method of claim 1, wherein said functional wild-type Pax4 is provided to said cell or explant culture as a Pax4 gene expression product or a functional fragment thereof.

4. The method of claim 3, wherein the Pax4 gene expression product is an mRNA or a protein.

5. The method of claim 1, wherein said Pax4 is provided as a nucleic acid molecule comprised in a vector.

6. The method of claim 5, wherein said vector is a viral vector.

7. The method of claim 6, wherein said viral vector is selected from the group consisting of a retroviral vector, a lentiviral vector and an adenoviral vector.

8. The method of claim 30, wherein said adenoviral vector is AdCMV or pHVAd2.

9. The method of claim 1, wherein the functional wild-type Pax4 comprises the wild-type Pax4 of mouse, rat or human.

10. The method of claim 1, wherein said functional, wild-type Pax4 is encoded by
(a) a nucleic acid molecule comprising a nucleic acid molecule encoding the polypeptide having the amino acid sequence as shown in SEQ ID NO: 2, 4, or 6;
(b) a nucleic acid molecule comprising a nucleic acid molecule having the DNA sequence as shown in SEQ ID NO: 1, 3, or 5;
(c) a nucleic acid molecule hybridizing to the complementary strand of nucleic acid molecules of (a) or (b) and encoding a functional wild-type Pax4; or
(d) a nucleic acid molecule being degenerate as a result of the genetic code to the nucleotide sequence of the nucleic acid molecule as defined in (c).

11. A pancreatic β-cell obtained by the in vitro method claim 1.

12. A pharmaceutical composition comprising a pancreatic β-cell obtained by the in vitro method of claim 1.

13-16. (canceled)

17. A method for treating a disorder characterized by insufficient pancreatic function in a subject comprising administering to said subject a pharmaceutical composition comprising a pancreatic β-cell as obtained by the in vitro method of claim 1.

18. The method of claim 17, wherein said subject is a human.

19. The method of claim 18, wherein said disorder is diabetes.

20-21. (canceled)

22. The method of claim 21, wherein said adenoviral vector is Ad2 or Ad5.

23. The method of claim 22, wherein said adenoviral vector comprises a DNA as shown in SEQ ID NOS: 12 or 14.

24. An adenoviral vector expressing a functional wild-type Pax4, wherein said adenoviral vector comprises SEQ ID NO: 15.

25. A kit comprising an adenoviral vector as defined in claim 30.

26. (canceled)

27. The method of claim 17, wherein said administering comprises transplantation or tissue replacement carried out in accordance with the Edmonton protocol.

28. The method of claim 27, wherein said administering comprises homo-transplantation or xenotransplantation.

29. The pancreatic β-cell of claim 11, wherein said pancreatic cell is a proliferating pancreatic cell.

30. The method of claim 7, wherein said viral vector is an adenoviral vector.


SEQUENCE LISTING (23 sequences)

SEQ ID NO: 1 - DNA, 1050 bp, Rattus norvegicus
atgcagcaggacggtctcagcagtgtgaatcagctggggggactctttgtgaatggccggccccttccactggacacccgacagcagattgtgcagctagcgataagagggatgcgaccctgtgacatctcacggagccttaaggtatctaacggctgtgtgagtaagatcctaggacgctactaccgcacaggagtcttggaacccaagggtattgggggaagcaaaccacgtctggccacacctgctgtggtggctcgaattgcccagctaaaggatgagtatccagctctttttgcctgggagatccaacgccagctttgtgctgaagggctttgtacccaggacaaggctcccagtgtatcctctatcaatcgagtccttcgggcactacaggaagaccagaggttacactggacacaacttagatctccagctgttttggctccagctcttcctagtccccacagtaactgtgaggctccccgaggtccccacccggggaccagccacaggaatcggactatcttctccccgggccaagctgaggcactggagaaagagttccagcgtgggcagtatccagattcagttgtccgtggaaagctggctgctgccacctctctgcctgaagacacagtgagggtctggttttccaacagaagagccaaatggcgcagacaagagaagttgaaatgggaaacacagatgccaggtgcttctcaggacctgatggtaccaaaagattctccagggatcatctctgcacagcagtcccctggcagtgtaccctcagcagccctgcctgtgctggaacaattgaatccttccttctgtcagctgtgctggggggcagtaccagacagatgttccagtgacaccacatcccaagcctgtctccaaccctactgggaatgccactccctccttcctgtggcttcttcctcatacatggaatttgcctggccctgcctcaccacccatcctgtgcatcatctgattggaggcccaggacaagcgccatcaacctattacttacactggccataa

SEQ ID NO: 2 - protein, 349 aa, Rattus norvegicus
MQQDGLSSVNQLGGLFVNGRPLPLDTRQQIVQLAIRGMRPCDISRSLKVSNGCVSKILGRYYRTGVLEPKGIGGSKPRLATPAVVARIAQLKDEYPALFAWEIQRQLCAEGLCTQDKAPSVSSINRVLRALQEDQRLHWTQLRSPAVLAPALPSPHSNCEAPRGPHPGTSHRNRTIFSPGQAEALEKEFQRGQYPDSVVRGKLAAATSLPEDTVRVWFSNRRAKWRRQEKLKWETQMPGASQDLMVPKDSPGIISAQQSPGSVPSAALPVLEQLNPSFCQLCWGAVPDRCSSDTTSQACLQPYWECHSLLPVASSSYMEFAWPCLTTHPVHHLIGGPGQAPSTYYLHWP

SEQ ID NO: 3 - DNA, 1050 bp, Mus musculus
atgcagcaggacggactcagcagtgtgaatcagctagggggactctttgtgaatggccggccccttcctctggacaccaggcagcagattgtgcagctagcaataagagggatgcgaccctgtgacatttcacggagccttaaggtatctaatggctgtgtgagcaagatcctaggacgctactaccgcacaggtgtcttggaacccaagtgtattgggggaagcaaaccacgtctggccacacctgctgtggtggctcgaattgcccagctaaaggatgagtaccctgctctttttgcctgggagatccaacaccagctttgcactgaagggctttgtacccaggacaaggctcccagtgtgtcctctatcaatcgagtacttcgggcacttcaggaagaccagagcttgcactggactcaactcagatcaccagctgtgttggctccagttcttcccagtccccacagtaactgtggggctccccgaggtccccacccaggaaccagccacaggaatcggactatcttctccccgggacaagccgaggcactggagaaagagtttcagcgtgggcagtatccagattcagtggcccgtgggaagctggctgctgccacctctctgcctgaagacacggtgagggtttggttttctaacagaagagccaaatggcgcaggcaagagaagctgaaatgggaagcacagctgccaggtgcttcccaggacctgacagtaccaaaaaattctccagggatcatctctgcacagcagtcccccggcagtgtaccctcagctgccttgcctgtgctggaaccattgagtccttccttctgtcagctatgctgtgggacagcaccaggcagatgttccagtgacacctcatcccaggcctatctccaaccctactgggactgccaatccctccttcctgtggcttcctcctcatatgtggaatttgcctggccctgcctcaccacccatcctgtgcatcatctgattggaggcccaggacaagtgccatcaacccattgctcaaactggccataa

SEQ ID NO: 4 - protein, 349 aa, Mus musculus
MQQDGLSSVNQLGGLFVNGRPLPLDTRQQIVQLAIRGMRPCDISRSLKVSNGCVSKILGRYYRTGVLEPKCIGGSKPRLATPAVVARIAQLKDEYPALFAWEIQHQLCTEGLCTQDKAPSVSSINRVLRALQEDQSLHWTQLRSPAVLAPVLPSPHSNCGAPRGPHPGTSHRNRTIFSPGQAEALEKEFQRGQYPDSVARGKLAAATSLPEDTVRVWFSNRRAKWRRQEKLKWEAQLPGASQDLTVPKNSPGIISAQQSPGSVPSAALPVLEPLSPSFCQLCCGTAPGRCSSDTSSQAYLQPYWDCQSLLPVASSSYVEFAWPCLTTHPVHHLIGGPGQVPSTHCSNWP

SEQ ID NO: 5 - DNA, 1049 bp, Homo sapiens
agggatcagcagcatgaaccagcttggggggctctttgtgaatggccggcccctgcctctggatacccggcagcagattgtgcggctagcagtcagtggaatgcggccctgtgacatctcacggatccttaaggtatctaatggctgtgtgagcaagatcctagggcgttactaccgcacaggtgtcttggagccaaagggcattgggggaagcaagccacggctggctacaccccctgtggtggctcgaattgcccagctgaagggtgagtgtccagccctctttgcctgggaaatccaacgccagctttgtgctgaagggctttgcacccaggacaagactcccagtgtctcctccatcaaccgagtcctgcgggcattacaggaggaccagggactaccgtgcacacggctcaggtcaccagctgttttggctccagctgtcctcactccccatagtggctctgagactccccggggtacccacccagggaccggccaccggaatcggactatcttctccccaagccaagcagaggcactggagaaagagttccagcgtgggcagtatcctgattcagtggcccgtggaaagctggctactgccacctctctgcctgaggacacggtgagggtctggttttccaacagaagagccaaatggcgtcggcaagagaagctcaagtgggaaatgcagctgccaggtgcttcccaggggctgactgtaccaagggttgccccaggaatcatctctgcacagcagtcccctggcagtgtgcccacagcagccctgcctgccctggaaccactgggtccctcctgctatcagctgtgctgggcaacagcaccagaaaggtgtctgagtgacaccccacctaaagcctgtctcaagccctgctggggccacttgcccccacagccgaattccctggactcaggactgctttgccttccttgcccttcctcccactgtcccctggccagtcttagtggctctcaggccctgctctggcctggctgcccactactgtatggcttggaatgaggca

SEQ ID NO: 6 - protein, 343 aa, Homo sapiens
MNQLGGLFVNGRPLPLDTRQQIVRLAVSGMRPCDISRILKVSNGCVSKILGRYYRTGVLEPKGIGGSKPRLATPPVVARIAQLKGECPALFAWEIQRQLCAEGLCTQDKTPSVSSINRVLRALQEDQGLPCTRLRSPAVLAPAVLTPHSGSETPRGTHPGTGHRNRTIFSPSQAEALEKEFQRGQYPDSVARGKLATATSLPEDTVRVWFSNRRAKWRRQEKLKWEMQLPGASQGLTVPRVAPGIISAQQSPGSVPTAALPALEPLGPSCYQLCWATAPERCLSDTPPKACLKPCWGHLPPQPNSLDSGLLCLPCPSSHCPLASLSGSQALLWPGCPLLYGLE

SEQ ID NO: 7 - DNA, 1050 bp, Mus musculus
atgcagcaggacggactcagcagtgtgaatcagctagggggactctttgtgaatggccggccccttcctctggacaccaggcagcagattgtgcagctagcaataagagggatgcgaccctgtgacatttcacggagccttaaggtatctaatggctgtgtgagcaagatcctaggacgctactaccgcacaggtgtcttggaacccaagtgtattgggggaagcaaaccacgtctggccacacctgctgtggtggctcgaattgcccagctaaaggatgagtaccctgctctttttgcctgggagatccaacaccagctttgcactgaagggctttgtacccaggacaaggctcccagtgtgtcctctatcaatcgagtactttgggcacttcaggaagaccagagcttgcactggactcaactcagatcaccagctgtgttggctccagttcttcccagtccccacagtaactgtggggctccccgaggtccccacccaggaaccagccacaggaatcggactatcttctccccgggacaagccgaggcactggagaaagagtttcagcgtgggcagtatccagattcagtggcccgtgggaagctggctgctgccacctctctgcctgaagacacggtgagggtttggttttctaacagaagagccaaatggcgcaggcaagagaagctgaaatgggaagcacagctgccaggtgcttcccaggacctgacagtaccaaaaaattctccagggatcatctctgcacagcagtcccccggcagtgtaccctcagctgccttgcctgtgctggaaccattgagtccttccttctgtcagctatgctgtgggacagcaccaggcagatgttccagtgacacctcatcccaggcctatctccaaccctactgggactgccaatccctccttcctgtggcttcctcctcatatgtggaatttgcctggccctgcctcaccacccatcctgtgcatcatctgattggaggcccaggacaagtgccatcaacccattgctcaaactggccataa

SEQ ID NO: 8 - protein, 349 aa, Mus musculus
MQQDGLSSVNQLGGLFVNGRPLPLDTRQQIVQLAIRGMRPCDISRSLKVSNGCVSKILGRYYRTGVLEPKCIGGSKPRLATPAVVARIAQLKDEYPALFAWEIQHQLCTEGLCTQDKAPSVSSINRVLWALQEDQSLHWTQLRSPAVLAPVLPSPHSNCGAPRGPHPGTSHRNRTIFSPGQAEALEKEFQRGQYPDSVARGKLAAATSLPEDTVRVWFSNRRAKWRRQEKLKWEAQLPGASQDLTVPKNSPGIISAQQSPGSVPSAALPVLEPLSPSFCQLCCGTAPGRCSSDTSSQAYLQPYWDCQSLLPVASSSYVEFAWPCLTTHPVHHLIGGPGQVPSTHCSNWP

SEQ ID NO: 9 - DNA, 1028 bp, Homo sapiens
atgaaccagcttggggggctctttgtgaatggccggcccctgcctctggatacccggcagcagattgtgcggctgcagtcagtggaatgcggccctgtgacatctcacggatccttaaggtatctaatggctgtgtgagcaagatcctagggcgttactaccgacaggtgtcttggagccaaagggcattgggggaagcaagccacggctggctacaccccctgtggtggctcgaattgcccagctgaagggtgagtgtccagccctctttgcctgggaaatccaacgccagctttgtgctgaagggctttgcacccaggacaagactcccagtgtctcctccatcaaccgagtcctgtgggcattacaggaggaccagggactaccgtgcacacggctcaggtcaccagctgttttggctccagctgtcctcactccccatagtggctctgagactccccggggtacccacccagggaccggccaccggaatcggactatcttctccccaagccaagcagaggcactggagaaagagttccagcgtgggcatatcctgattcagtggcccgtggaaagctggctactgccacctctctgcctgaggacacggtgagggtctggttttccaacagaagagccaaatggcgtcggcaagagaagctcaagtgggaaatgcagctgccaggtgcttcccaggggctgactgtaccaagggttgccccaggaatcatctctgcacagcagtcccctggcagtgtgcccacagcagccctgcctgccctggaaccctgggtccctcctgctatcagctgtgctgggcaacagcaccagaaaggtgtctgagtgacaccccacctaaagcctgtctcaagccctgctggggccacttgcccccacagccgaattccctggactcaggactgctttgccttccttgcccttcctcccactgtcccctggccagtcttagtggctctcaggccctgctctggcctggctgcccactactgtatggcttggaatga

SEQ ID NO: 10 - protein, 343 aa, Homo sapiens
MNQLGGLFVNGRPLPLDTRQQIVRLAVSGMRPCDISRILKVSNGCVSKILGRYYRTGVLEPKGIGGSKPRLATPPVVARIAQLKGECPALFAWEIQRQLCAEGLCTQDKTPSVSSINRVLWALQEDQGLPCTRLRSPAVLAPAVLTPHSGSETPRGTHPGTGHRNRTIFSPSQAEALEKEFQRGQYPDSVARGKLATATSLPEDTVRVWFSNRRAKWRRQEKLKWEMQLPGASQGLTVPRVAPGIISAQQSPGSVPTAALPALEPLGPSCYQLCWATAPERCLSDTPPKACLKPCWGHLPPQPNSLDSGLLCLPCPSSHCPLASLSGSQALLWPGCPLLYGLE

SEQ ID NO: 11 - DNA, 4437 bp, Artificial Sequence
  Description of Artificial Sequence  Synthetic Primer
taactataacggtcctaaggtagcgaaagctcagatctggatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcgagctcggtacccggggatcctctagtcagctgacgcgtgctagcgcggccgcatcgataagcttgtcgacgatatctctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccaggaagctcctctgtgtcctcataaaccctaacctcctctacttgagaggacattccaatcataggctgcccatccaccctctgtgtcctcctgttaattaggtcacttaacaaaaaggaaattgggtaggggtttttcacagaccgctttctaagggtaattttaaaatatctgggaagtcccttccactgctgtgttccagaagtgttggtaaacagcccacaaatgtcaacagcagaaacatacaagctgtcagctttgcacaagggcccaacaccctgctcatcaagaagcactgtggttgctgtgttagtaatgtgcaaaacaggaggcacattttccccacctgtgtaggttccaaaatatctagtgttttcatttttacttggatcaggaacccagcactccactggataagcattatccttatccaaaacagccttgtggtcagtgttcatctgctgactgtcaactgtagcattttttggggttacagtttgagcaggatatttggtcctgtagtttgctaacacaccctgcagatctgaattcatctatgtcgggtgcggagaaagaggtaatgaaatggcattatgggtattatgggtctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatcctccggcgttcagcctgtgccacagccgacaggatggtgaccaccatttgccccatatcaccgtcggtactgatcccgtcgtcaataaaccgaaccgctacaccctgagcatcaaactcttttatcagttggatcatgtcggcggtgtcgcggccaagacggtcgagcttcttcaccagaatgacatcaccttcctccaccttcatcctcagcaaatccagcccttcccgatctgttgaactgccggatgccttgtcggtaaagatgcggttagcttttacccctgcatctttgagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagattattgaagcatttatcagggttattgtctcatgagcggatacatatttgaatgtatttagaaaaataaacaaataggggttccgcgcacatttccccgaaaagtgccacctgacg

SEQ ID NO: 12 - DNA, 32705 bp, Artificial Sequence
  Description of Artificial Sequence  Synthetic Primer
cgtaactataacggtcctaaggtagcgaaaattatttaaatatctatgtcgggtgcggagaaagaggtaatgaaatggcatcgactcgaagatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttataagttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttacatcgttaattaacgatttcgaacccggggtaccgaattcctcgagtctagaggagcatgcgacgtcgcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctagttaattaacgttaattaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatctctagcat

SEQ ID NO: 13 - DNA, 736 bp, Artificial Sequence
  Description of Artificial Sequence  Synthetic Primer
atgaaccagcttggggggctctttgtgaatggccggcccctgcctctggatacccggcagcagattgtgcggctagcagtcagtggaatgcggccctgtgacatctcacggatccttaaggtatctaatggctgtgtgagcaagatcctagggcgttactaccgcacaggtgtcttggagccaaagggcattgggggaagcaagccacggctggctacaccccctgtggtggctcgaattgcccagctgaagggtgagtgtccagccctctttgcctgggaaatccaacgccagctttgtgctgaagggctttgcacccaggacaagactcccagtgtctcctccatcaaccgagtcctgcgggcattacaggaggaccagggactaccgtgcacacggctcaggtcaccagctgttttggctccagctgtcctcactccccatagtggctctgagactccccggggtacccacccagggaccggccaccggaatcggactatcttctccccaagccaagcagaggcactggagaaagagttccagcgtgggcagtatcctgattcagtggcccgtggaaagctggctactgccacctctctgcctgaggacacggtgagggtctggttttccaacagaagagccaaatggcgtcggcaagagaagctcaagtgggaaatgcagctgccaggtgcttcccaggggctgactgtaccaagggttgccccaggaatca

SEQ ID NO: 14 - DNA, 4714 bp, Artificial Sequence
  Description of Artificial Sequence  Synthetic Primer
tcccttccagctctctgccccttttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatctctagcatcgatgtcgaggatccctcgagtctagagatatcgaattcaagcttgtcgactcgaagatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctctcgatcgaggcggccgcagatctcgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgggatcaattcttaattaa

SEQ ID NO: 15 - DNA, 7064 bp, Artificial Sequence
  Description of Artificial Sequence  Synthetic Primer
tcccttccagctctctgccccttttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatctctagcatcgatgtcgaggatccctcgagtctagagatggccgcgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctcgtgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatccggtcgcgcgaattcgagctcggtacccggggatcccccgatcgcgactgtgaggagtaccagtgtgaagcatgcagcaggacggactcagcagtgtgaatcagctagggggactctttgtgaatggccggccccttcctctggacaccaggcagcagattgtgcagctagcaataagagggatgcgaccctgtgacatttcacggagccttaaggtatctaatggctgtgtgagcaagatcctaggacgctactaccgcacaggtgtcttggaacccaagtgtattgggggaagcaaaccacgtctggccacacctgctgtggtggctcgaattgcccagctaaaggatgagtaccctgctctttttgcctgggagatccaacaccagctttgcactgaagggctttgtacccaggacaaggctcccagtgtgtcctctatcaatcgagtacttcgggcacttcaggaagaccagagcttgcactggactcaactcagatcaccagctgtgttggctccagttcttcccagtccccacagtaactgtggggctccccgaggcccccacccaggaaccagccacaggaatcgggctatcttctccccgggacaagccgaggcactggagaaagagtttcagcgtgggcagtatccagattcagtggcccgtgggaagctggctgctgccacctctctgcctgaagacacggtgagggtttggttttctaacagaagagccaaatggcgcaggcaagagaagctgaaatgggaagcacagctgccaggtgcttcccaggacctgacagtaccaaaaaattctccagggatcatctctgcacagcagtcccccggcagtgtaccctcagctgccttgcctgtgctggaaccattgagtccttccttctgtcagctatgctgtgggacagcaccaggcagatgttccagtgacacctcatcccaggcctatctccaaccctactgggactgccaatccctccttcctgtggcttcctcctcatatgtggaatttgcctggccctgcctcaccacccatcctgtgcatcatctgattggaggcccaggacaagtgccatcaacccattgctcaaactggccataaccgcggaattcgatatcaagcttgggatctttgtgaaggaaccttacttctgtggtgtgacataattggacaaactacctacagagatttaaagctctaaggtaaatataaaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcgcggccatcgaattcaagcttgtcgactcgaagatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctctcgatcgaggcggccgcagatctcgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgggatcaattcttaattaa

SEQ ID NO: 16 - DNA, 35474 bp, Artificial Sequence
  Description of Artificial Sequence  Synthetic Primer
cgtaactataacggtcctaaggtagcgaaagctcagatctggatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcgagctcggtacccggggatcctctagtcagctgacgcgtgctagcgcggccgcatcgataagcttgtcgacgatgcttggtaccgagctcggatccactagtccagtgtggtggaattcgatttgaagatgaaccagcttggggggctctttgtgaatggccggcccctgcctctggatacccggcagcagattgtgcggctagcagtcagtggaatgcggccctgtgacatctcacggatccttaaggtatctaatggctgtgtgagcaagatcctagggcgttactaccgcacaggtgtcttggagccaaagggcattgggggaagcaagccacggctggctacaccccctgtggtggctcgaattgcccagctgaagggtgagtgtccagccctctttgcctgggaaatccaacgccagctttgtgctgaagggctttgcacccaggacaagactcccagtgtctcctccatcaaccgagtcctgcgggcattacaggaggaccagggactaccgtgcacacggctcaggtcaccagctgttttggctccagctgtcctcactccccatagtggctctgagactccccggggtacccacccagggaccggccaccggaatcggactatcttctccccaagccaggcagaggcactggagaaagagttccagcgtgggcagtatcctgattcagtggcccgtggaaagctggctactgccacctctctgcctgaggacacggtgagggtctggttttccaacagaagagccaaatggcgtcggcaagagaagctcaagtgggaaatgcagctgccaggtgcttcccaggggctgactgtaccaagggttgccccaggaatcatctctgcacagcagtcccctggcagtgtgcccacagcagccctgcctgccctggaaccactgggtccctcctgctatcagctgtgctgggcaacagcaccagaaaggtgtctgagtgacaccccacctaaagcctgtctcaagccctgctggggccacttgcccccacagccgaattccctggactcaggactgctttgccttccttgcccttcctcccactgtcccctggccagtcttagtggctctcaggccctgctctggcctggctgcccactactgtatggcttggaaatcactagtgaattctgcagatatccagcacagtggcggccgctcgagtctagagggcccttcgaacaaaaactcatctcagaagaggatctgaatatgcataccggtcatcatcaccatcaccattgagttatctctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccaggaagctcctctgtgtcctcataaaccctaacctcctctacttgagaggacattccaatcataggctgcccatccaccctctgtgtcctcctgttaattaggtcacttaacaaaaaggaaattgggtaggggtttttcacagaccgctttctaagggtaattttaaaatatctgggaagtcccttccactgctgtgttccagaagtgttggtaaacagcccacaaatgtcaacagcagaaacatacaagctgtcagctttgcacaagggcccaacaccctgctcatcaagaagcactgtggttgctgtgttagtaatgtgcaaaacaggaggcacattttccccacctgtgtaggttccaaaatatctagtgttttcatttttacttggatcaggaacccagcactccactggataagcattatccttatccaaaacagccttgtggtcagtgttcatctgctgactgtcaactgtagcattttttggggttacagtttgagcaggatatttggtcctgtagtttgctaacacaccctgcagatctgaattcatctatgtcgggtgcggagaaagaggtaatgaaatggcatcgactcgaagatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttataagttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttacatcgttaattaacgatttcgaacccggggtaccgaattcctcgagtctagaggagcatgcgacgtcgcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctagttaattaacgttaattaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatctctagcat

SEQ ID NO: 17 - DNA, 19 bp, Artificial Sequence
  Description of Artificial Sequence  Synthetic Primer
cgagtactttgggcacttc

SEQ ID NO: 18 - DNA, 19 bp, Artificial Sequence
  Description of Artificial Sequence  Synthetic Primer
gaagtgcccaaagtactcg

SEQ ID NO: 19 - DNA, 30 bp, Artificial Sequence
  Description of Artificial Sequence  Synthetic Primer
gaacaaaaactcatctcagaagaggatctg

SEQ ID NO: 20 - DNA, 33 bp, Artificial Sequence
  Description of Artificial Sequence  Synthetic Primer
aatatgcataccggtcatcatcaccatcaccat

SEQ ID NO: 21 - DNA, 21 bp, artificial sequence
  siRNA siPD21
gcaggcaagagaagctgaaat

SEQ ID NO: 22 - DNA, 21 bp, artificial sequence
  siRNA siHD21
ggctcgaattgcccagctaaa

SEQ ID NO: 23 - DNA, 29 bp, artificial sequence
  siRNA siPD29
ggctcgaattgcccagctaaaggatgagt